SEQUENCE LISTING (8 sequences)

SEQ ID NO: 1 - DNA, 31 bp, Artificial Sequence
  Synthetic
gttttcttttgaacatcttctctttcatcta

SEQ ID NO: 2 - DNA, 31 bp, Artificial Sequence
  Synthetic
tagatgaaagagaagatgttcaaaagaaaac

SEQ ID NO: 3 - DNA, 30 bp, Artificial Sequence
  Synthetic
gtatctctattattcatcgtcttattcttc

SEQ ID NO: 4 - DNA, 31 bp, Artificial Sequence; identical to SEQ ID NO: 2
  Synthetic

SEQ ID NO: 5 - DNA, 31 bp, Artificial Sequence
  Synthetic
atctactttctcttctacaagttttcttttg

SEQ ID NO: 6 - DNA, 31 bp, Artificial Sequence
  Synthetic
caaaagaaaacttgtagaagagaaagtagat

SEQ ID NO: 7 - DNA, 20 bp, Artificial Sequence
  Synthetic
tacctaagcctcctggagca

SEQ ID NO: 8 - DNA, 23 bp, Artificial Sequence
  Synthetic
cttttggcagtttttgccctgta

(19) United States
(12) Patent Application Publication
(10) Pub. No.: US 2024/0209360 A1
(43) Pub. Date: Jun. 27, 2024

(54) PRODUCTS AND METHODS FOR INDUCING EXON 2 SKIPPING OF THE DMD GENE IN TREATING MUSCULAR DYSTROPHY

(71) Applicant: RESEARCH INSTITUTE AT NATIONWIDE CHILDREN'S HOSPITAL, Columbus, OH (US)

(72) Inventors: Kevin Flanigan, Columbus, OH (US); Nicolas Sebastien Wein, Columbus, OH (US); Tabatha Simmons, Columbus, OH (US); Adeline Vulin-Chaffiol, Columbus, OH (US)

(21) Appl. No.: 18/287,682

(22) PCT Filed: Apr. 27, 2022
(86) PCT No.: PCT/US22/26515

§ 371 Date: Oct. 20, 2023

Related U.S. Application Data
(60) Provisional application No. 63/180,355, filed on Apr. 27, 2021.

(51) Int. Cl. C12N 15/113; A61K 31/573; A61P 21/00

(52) U.S. Cl. CPC C12N 15/113; A61K 31/573; A61P 21/00; C12N 2310/11; C12N 2310/3233; C12N 2310/3513; C12N 2320/33

(57) ABSTRACT

Products and methods for treating or preventing muscular dystrophies in patients with mutations in the 5′ end of their DMD gene are provided. In some aspects, oligonucleotides, antisense phosphorodiamidate morpholino oligomers (PMO), and antisense cell penetrating peptide-conjugated PMOs (PPMOs) are provided for skipping exon 2 of the DMD gene. These oligonucleotides and oligomers can selectively suppress mutant forms of the dystrophin protein while allowing a functional form of the dystrophin protein to be expressed in sufficient quantity to retain its function in the cell. The oligonucleotides or oligomers can regulate or restore expression of transcripts of the DMD gene and a functional form of the dystrophin protein. Methods comprising administering the oligonucleotides. PMO, and PPMO targeting the DMD gene are provided. The products and methods are used for treating, ameliorating and/or preventing muscular dystrophies, such as Duchenne Muscular Dystrophy or Becker Muscular Dystrophy.

### CROSS-REFERENCE TO RELATED APPLICATIONS

The present application claims the priority benefit of U.S. Provisional Patent Application No. 63/180,355, filed Apr. 27, 2021, hereby incorporated by reference in its entirety.

### STATEMENT OF GOVERNMENT INTEREST

This invention was made with government support under grant no. AR070604 awarded by the National Institutes of Health. The government has certain rights in the invention.

### INCORPORATION BY REFERENCE OF THE SEQUENCE LISTING

This application contains, as a separate part of disclosure, a Sequence Listing in computer-readable form (Filename: 56492_SeqListing.txt; 1,778 bytes—ASCII text file created Apr. 26, 2022) which is incorporated by reference herein in its entirety.

### FIELD

The disclosure relates to the field of gene therapy for the treatment and/or prevention of muscular dystrophy. More particularly, the disclosure provides products and methods for treating or preventing muscular dystrophies in patients with 5′ mutation of the DMD gene. The disclosure provides oligonucleotides and antisense oligonucleotide constructs comprising peptide-conjugated oligonucleotides. In some aspects, such antisense oligonucleotide constructs are peptide-linked phosphorodiamidate morpholino oligonucleotides (PMO) or cell penetrating peptide-conjugated PMOs (PPMOs) for antisense-mediated exon-skipping to skip frame-disrupting exon(s) and allow functional dystrophin protein expression by restoring the reading frame. More specifically, such PMOs and PPMOs are used for skipping exon 2 of the DMD gene involved in muscular dystrophy (MD) disease. The oligonucleotides (or antisense oligonucleotide constructs) can selectively suppress mutant forms of the dystrophin protein while allowing a functional form of the dystrophin protein to be expressed in sufficient quantity to retain its function in the cell. The oligonucleotides can regulate or restore expression of transcripts of the DMD gene and a functional form of the dystrophin protein in cells of the muscle. The products and methods are used for treating, ameliorating and/or preventing MD diseases, such as Duchenne Muscular Dystrophy or Becker Muscular Dystrophy.

### BACKGROUND

Muscular dystrophies (MDs) are a group of genetic degenerative diseases primarily affecting voluntary muscles. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

The MDs are a group of diseases without identifiable treatment that gravely impact individuals, families, and communities. The costs are incalculable. Individuals suffer emotional strain and reduced quality of life associated with loss of self-esteem. Extreme physical challenges resulting from loss of limb function creates hardships in activities of daily living. Family dynamics suffer through financial loss and challenges to interpersonal relationships. Siblings of the affected feel estranged, and strife between spouses often leads to divorce, especially if responsibility for the muscular dystrophy can be laid at the feet of one of the parental partners. The burden of quest to find a cure often becomes a life-long, highly focused effort that detracts and challenges every aspect of life. Beyond the family, the community bears a financial burden through the need for added facilities to accommodate the handicaps of the muscular dystrophy population in special education, special transportation, and costs for recurrent hospitalizations to treat recurrent respiratory tract infections and cardiac complications. Financial responsibilities are shared by state and federal governmental agencies extending the responsibilities to the taxpaying community.

One form of MD is Duchenne Muscular Dystrophy (DMD). DMD, an X-linked degenerative muscle disorder, is the most common severe childhood form of muscular dystrophy affecting around 1:5200 male births (Mendell et al., Ann Neurol 71, 304-313 (2012)). Symptoms of generalized muscle weakness first appear at ages 3-5 and progress into a loss of ambulation by age 13, with death typically occurring in the third decade of life due to cardiomyopathy or respiratory insufficiency (Passamano et al., Acta Myol 31, 121-125 (2012); Duchenne, The Pathology of Paralysis with Muscular Degeneration (Paralysie Myosclerotique), or Paralysis with Apparent Hypertrophy. Br Med J 2, 541-542 (1867)). DMD is caused by mutations that disrupt the open reading frame in the DMD gene, which encodes dystrophin (Juan-Mateu et al., PLOS One 10, e0135189 (2015)), a large (427 kDa) multifunctional protein that is localized at the subsarcolemmal region of myofibers, where it plays an important role in protecting the sarcolemma from mechanical damage caused by muscle contraction (Petrof et al., Proc Natl Acad Sci USA 90, 3710-3714 (1993)).

Another form of MD is Becker Muscular Dystrophy (BMD). BMD is a milder allelic disorder which results from the presence of a partially functional dystrophin protein occurring from mutations that maintain an open reading frame (ORF) (Wein et al., Nature Medicine 20, 992-1000 (2014); Monaco, Trends Biochem Sci 14, 412-415 (1989)). BMD, like DMD, is a genetic disorder that gradually makes the body's muscles weaker and smaller. BMD affects the muscles of the hips, pelvis, thighs, and shoulders, as well as the heart, but is known to cause less severe problems than DMD. Because of the variety of in-frame mutations resulting in a variety of partially functional proteins, BMD has a broad phenotypic spectrum with, for example, loss of ambulation ranging from the late teenage years to late adulthood.

Promising therapeutic approaches to DMD are based on the replacement of a functional version of DMD, or its repair at the DNA or pre-mRNA level. Both approaches aim at restoration of an open reading frame, leading to expression of a partially function, BMD-like dystrophin. Gene replacement trials using modified adeno-associated viruses (AAVs) have been reported (Muzyczka, Curr Top Microbiol Immunol 158, 97-129 (1992); Carter, Mol Ther 10, 981-989 (2004); Samulski et al., Annu Rev Virol 1, 427-451 (2014)), but transgene packaging capacity of AAV is limited to ˜5 kb. Because the DMD cDNA is 11.4 kb, current viral vectors make use of one of several internally-deleted but in-frame microdystrophin CDNAs (Duan, Mol Ther 26, 2337-2356 (2018)). An alternate approach is to restore the mRNA reading frame by delivering an antisense sequence that binds to key exon definition elements in the pre-mRNA, inhibiting the recognition of a specific exon by the spliceosome, leading to exclusion of the target exon from the mature RNA. Such antisense sequences can consist of antisense oligonucleotides (AONs), or phoshphorodiamidate morpholino oligomers (PMO), such as eteplirsen, the first such therapy approved by the FDA for treatment of DMD due to mutations amenable to skipping of exon 51 (Barthelemy et al., Neuromuscul Disord 28, 803-824 (2018); Wein et al., Pediatr Clin North Am 62, 723-742 (2015); Alfano et al., Medicine (Baltimore) 98, e15858 (2019)).

Despite many lines of research following the identification of the DMD gene, treatment options are limited. There thus remains a need in the art for treatments for MDs, including DMD and BMD, including treatments for one or more mutations of the DMD gene.

### SUMMARY

The disclosure provides products and methods for preventing disease, delaying the progression or severity of disease, and/or treating disease in patients with one or more mutations in patients with 5′ mutation of the DMD gene. In some aspects, the disclosure provides products and methods for treating a mutation in any one or more of exons 1-5. In some aspects, the disclosure provides products and methods to induce skipping of exon 2 of the DMD gene.

The disclosure provides an oligonucleotide comprising a nucleotide sequence selected from the group consisting of:
(a) a nucleotide sequence comprising at least 97%, at least 98%, at least 99% identity to the sequence set forth in any one of SEQ ID NOs: 1 and 2; and
(b) the nucleotide sequence comprising the sequence set forth in any one of SEQ ID NOs: 1 and 2.

The disclosure provides an antisense oligonucleotide construct comprising a peptide conjugated to an oligonucleotide comprising a nucleotide sequence selected from the group consisting of:
(a) a nucleotide sequence comprising at least 80% identity to the sequence set forth in any one of SEQ ID NOs: 1 and 2;
(b) a nucleotide sequence complementary to the nucleotide sequence comprising at least 80% identity to the sequence set forth in any one of SEQ ID NOs: 1 and 2;
(c) a nucleotide sequence comprising the sequence set forth in any one of SEQ ID NOs: 1 and 2;
(d) a nucleotide sequence complementary to the nucleotide sequence comprising the sequence set forth in any one of SEQ ID NOs: 1 and 2; and
(e) a nucleotide sequence which binds to the sequence set forth in SEQ ID NO: 4.

In some aspects, the antisense oligonucleotide construct is a phosphorodiamidate morpholino oligomer (PMO) or a peptide-conjugated PMO (PPMO).

The disclosure provides a composition comprising an oligonucleotide comprising a nucleotide sequence selected from the group consisting of:
(a) a nucleotide sequence comprising at least 97%, at least 98%, at least 99% identity to the sequence set forth in any one of SEQ ID NOs: 1 and 2; and
(b) the nucleotide sequence comprising the sequence set forth in any one of SEQ ID NOs: 1 and 2; and a carrier, diluent, excipient, and/or adjuvant.

The disclosure provides a composition comprising an antisense oligonucleotide construct comprising a peptide conjugated to an oligonucleotide comprising a nucleotide sequence selected from the group consisting of:
(a) a nucleotide sequence comprising at least 80% identity to the sequence set forth in any one of SEQ ID NOs: 1 and 2;
(b) a nucleotide sequence complementary to the nucleotide sequence comprising at least 80% identity to the sequence set forth in any one of SEQ ID NOs: 1 and 2;
(c) a nucleotide sequence comprising the sequence set forth in any one of SEQ ID NOs: 1 and 2;
(d) a nucleotide sequence complementary to the nucleotide sequence comprising the sequence set forth in any one of SEQ ID NOs: 1 and 2; and
(e) a nucleotide sequence which binds to the sequence set forth in SEQ ID NO: 4; and a carrier, diluent, excipient, and/or adjuvant.

The disclosure provides a method of treating, preventing and/or ameliorating a muscular dystrophy in a subject in need thereof comprising the step of administering to the subject an effective amount of
(i) an oligonucleotide comprising a nucleotide sequence selected from the group consisting of:
   (a) a nucleotide sequence comprising at least 97%, at least 98%, at least 99% identity to the sequence set forth in any one of SEQ ID NOs: 1 and 2; and
   (b) the nucleotide sequence comprising the sequence set forth in any one of SEQ ID NOs: 1 and 2;
(ii) an antisense oligonucleotide construct comprising a peptide conjugated to an oligonucleotide comprising a nucleotide sequence selected from the group consisting of:
   (a) a nucleotide sequence comprising at least 80% identity to the sequence set forth in any one of SEQ ID NOs: 1 and 2;
   (b) a nucleotide sequence complementary to the nucleotide sequence comprising at least 80% identity to the sequence set forth in any one of SEQ ID NOs: 1 and 2;
   (c) a nucleotide sequence comprising the sequence set forth in any one of SEQ ID NOs: 1 and 2;
   (d) a nucleotide sequence complementary to the nucleotide sequence comprising the sequence set forth in any one of SEQ ID NOs: 1 and 2; and
   (e) a nucleotide sequence which binds to the sequence set forth in SEQ ID NO: 4; and
(iii) a composition comprising any of the oligonucleotides or the antisense oligonucleotide constructs described herein and a carrier, diluent, excipient, and/or adjuvant.

In some aspects, the muscular dystrophy results from a 5′ mutation in the DMD gene. In some aspects, the 5′ mutation is a mutation involving any one of exons 1-5. In some aspects, the 5′ mutation is an exon 2 duplication.

In some aspects, the administering is via a systemic route. In some aspects, the systemic route is by injection, infusion or implantation. In some aspects, the injection is an intravenous injection.

In some aspects, the antisense oligonucleotide construct is a PMO or PPMO. In some aspects, the PMO or PPMO is administered to the subject at a dose of about 1 to about 100 mg/kg.

In some aspects, the muscular dystrophy is Duchenne Muscular Dystrophy or Becker Muscular Dystrophy.

In some aspects, the level of functional dystrophin gene expression or protein expression in a cell of the subject is increased after administering the oligonucleotide, the antisense oligonucleotide construct, or the composition as compared to the level of functional dystrophin gene expression or protein expression before administering the oligonucleotide, the antisense oligonucleotide construct, or the composition.

In some aspects, the expression of functional dystrophin in the cell is detected by measuring the dystrophin protein level by Western blot, immunofluorescence, or immunohistochemistry in muscle biopsied before and after administering the oligonucleotide, the antisense oligonucleotide construct, or the composition.

In some aspects, the level of serum creatinine kinase is decreased after administering the oligonucleotide, the antisense oligonucleotide construct, or the composition as compared to the level of serum creatinine kinase before administering the oligonucleotide, the antisense oligonucleotide construct, or the composition.

In some aspects, a method of the disclosure results in improved muscle strength, improved muscle function, improved mobility, improved stamina, or a combination of two or more thereof in the subject.

In some aspects, muscular dystrophy progression in the subject is delayed or wherein muscle function in the subject is improved after administering the oligonucleotide, the antisense oligonucleotide construct, or the composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

In some aspects, a method of the disclosure further comprises administering a second or combination therapy. In some aspects, the second or combination therapy is the administration of a glucocorticoid.

The disclosure also provides us of
(i) an oligonucleotide comprising a nucleotide sequence selected from the group consisting of:
   (a) a nucleotide sequence comprising at least 97%, at least 98%, at least 99% identity to the sequence set forth in any one of SEQ ID NOs: 1 and 2; and
   (b) the nucleotide sequence comprising the sequence set forth in any one of SEQ ID NOs: 1 and 2;
(ii) an antisense oligonucleotide construct comprising a peptide conjugated to an oligonucleotide comprising a nucleotide sequence selected from the group consisting of:
   (a) a nucleotide sequence comprising at least 80% identity to the sequence set forth in any one of SEQ ID NOs: 1 and 2;
   (b) a nucleotide sequence complementary to the nucleotide sequence comprising at least 80% identity to the sequence set forth in any one of SEQ ID NOs: 1 and 2;
   (c) a nucleotide sequence comprising the sequence set forth in any one of SEQ ID NOs: 1 and 2;
   (d) a nucleotide sequence complementary to the nucleotide sequence comprising the sequence set forth in any one of SEQ ID NOs: 1 and 2; and
   (e) a nucleotide sequence which binds to the sequence set forth in SEQ ID NO: 4; and
(iii) a composition comprising any of the oligonucleotides or the antisense oligonucleotide constructs described herein and a carrier, diluent, excipient, and/or adjuvant for the preparation of a medicament for the treatment of a muscular dystrophy, or for treating a muscular dystrophy in a subject in need thereof.

In some aspects, the muscular dystrophy results from a 5′ mutation in the DMD gene. In some aspects, the 5′ mutation is a mutation involving any one of exons 1-5. In some aspects, the 5′ mutation is an exon 2 duplication.

In some aspects, the administering is via a systemic route. In some aspects, the systemic route is by injection, infusion or implantation. In some aspects, the injection is an intravenous injection.

In some aspects, the antisense oligonucleotide construct is a PMO or PPMO. In some aspects, the PMO or PPMO is administered to the subject at a dose of about 1 to about 100 mg/kg.

In some aspects, the muscular dystrophy is Duchenne Muscular Dystrophy or Becker Muscular Dystrophy.

In some aspects, the level of functional dystrophin gene expression or protein expression in a cell of the subject is increased after administering the oligonucleotide, the antisense oligonucleotide construct, or the composition as compared to the level of functional dystrophin gene expression or protein expression before administering the oligonucleotide, the antisense oligonucleotide construct, or the composition.

In some aspects, the expression of functional dystrophin in the cell is detected by measuring the dystrophin protein level by Western blot, immunofluorescence, or immunohistochemistry in muscle biopsied before and after administering the oligonucleotide, the antisense oligonucleotide construct, or the composition.

In some aspects, the level of serum creatinine kinase is decreased after administering the oligonucleotide, the antisense oligonucleotide construct, or the composition as compared to the level of serum creatinine kinase before administering the oligonucleotide, the antisense oligonucleotide construct, or the composition.

In some aspects, the use results in improved muscle strength, improved muscle function, improved mobility, improved stamina, or a combination of two or more thereof in the subject.

In some aspects, muscular dystrophy progression in the subject is delayed or wherein muscle function in the subject is improved after administering the oligonucleotide, the antisense oligonucleotide construct, or the composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

In some aspects, the use further comprises administering a second or combination therapy. In some aspects, the second or combination therapy is the administration of a glucocorticoid.

Other features and advantages of the disclosure will become apparent from the following description of the drawing and the detailed description. It should be understood, however, that the drawing, detailed description, and the examples, while indicating aspects of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

FIG. 1 shows representative images of PPMO-induced DMD exon 2 skipping in quadriceps. Exon 2 exclusion in Dup2 mice (n=5-6) treated for 7 days with a dose escalation of PPMO-A1 (20, 40 and 80 mg/kg), PPMO-Scr (80 mg/kg) or saline (Sal), and C57BI/6 (BI6) (n=5) injected with saline as a control. Only right side of quadriceps (R. Quad) in all groups was used in RT-PCR analysis. Dup2, wild type (WT) and Del2 mRNA transcripts are represented as 340 bp, 278 bp and 216 bp bands, respectively. RT(−) and PCR(−) are negative controls, in which water was used as a template for RT and PCR steps to ensure that the tested samples were free of DNA contamination and non-specific amplification. Ladder, 1 kb Plus DNA ladder.

FIG. 2A-B shows transcriptional dose-response for PPMO-A1. FIG. 2A shows quantification analysis of exon 2 skipping in right quadriceps (R. Quad), right gastrocnemius (R. Gas), right tibialis anterior (R. TA), diaphragm (Dia) and heart from Dup2 mice treated with doses of PPMO-A1 ranging from 20 to 80 mg/kg (20, 40, 80), PPMO-Scr (Scr) at the dose of 80 mg/kg or saline diluent (Sal). C57BI/6 (BI6) mice injected with saline used as controls (n=4-7 per tissue). Dup2 band is shown in white, whereas therapeutic transcripts, defined as wild type (WT) and Del2 dystrophin transcripts with 1 or 0 copies of exon 2, are shown in grey and black, respectively. BI6 mice are represented as 100% WT transcript. Data are presented as mean±SEM. FIG. 2B shows linear regression analysis of the percent therapeutic transcript (WT+Del2) vs. PPMO-A1 dose for each tissue (n=4-7 per tissue); **p<0.01; ***p<0.001; ****p<0.0001.

FIG. 3 shows transcriptional analysis confirms DMD exon 2 exclusion in PPMO-A1 time-course study. Right quadriceps (R. Quad), right gastrocnemius (R. Gas), right tibialis anterior (R. TA), diaphragm (Dia), and hearts from Dup2 mice systemically injected with PPMO-A1 (80) or PPMO-Scr (Scr) at the dose of 80 mg/kg, or saline (Sal) showing skipping of DMD exon 2 at 7, 15, and 30 days after treatment. Dup2 band is shown in white, whereas therapeutic transcripts, WT and Del2, are shown in grey and black, respectively. C57BI/6 (BI6) mice are displayed as 100% WT transcript. Data are represented as mean±SEM from each group in all tissues (n=4-7 per group). Where appropriate, the total therapeutic transcripts (WT+Del2) were compared for 80 mg/kg PPMO-A1 (80) and control groups with a Welch's ANOVA and Dunnett's T3 multiple comparison test. Groups without variation—all BI6 and some Sal and Scr—were excluded from statistical analysis. Statistical comparison markers indicate which groups were analyzed. ns=not significant; *p<0.05; **p<0.01.

FIG. 4A-D shows dystrophin expression in muscles 15, 30 and 60 days following PPMO-A1 injection. Immunofluorescence (IF) quantification showing the percentage of dystrophin-positive fibers in (FIG. 4A) left tibialis anterior (L. TA) and (FIG. 4B) diaphragm from Dup2 mice injected with either PPMO-A1 or PPMO-Scr at the dose of 80 mg/kg, or saline (Sal) at all tested timepoints (15, 30, and 60 days after treatment). Wild type (BI6) mice injected with saline used as controls. The dystrophin-positive fiber content is plotted for each treatment group and timepoint, with individual data points showing the percent dystrophin-positive fibers for each mouse. The data presented as mean±SEM, n=3-6 per tissue. *p<0.05; **p<0.01; ***p<0.001; ****p<0.0001. Statistically significant differences identified by Welch's ANOVA with Dunnett's T3 multiple comparison test. Representative whole-section images of (FIG. 4C) left TA (L. TA) and (FIG. 4D) diaphragm (Dia) (2 mm segments) with laminin in green and dystrophin in red displaying localization and intensity of dystrophin signal in treated mice. Color-coded heatmaps of each tissue display the percent of the perimeter with dystrophin-positive pixels for each muscle fiber. Fibers with ≥70% dystrophin-positive perimeter are considered overall positive for dystrophin. Scale bars=500 μm. Color key in bottom corner shows color to percent conversion for dystrophin-positive perimeter heatmaps.

FIG. 5A-D shows dystrophin immunofluorescence intensity in muscle after PPMO-A1 treatment. Cumulative histograms of muscle fiber dystrophin intensities in (FIG. 5A) left TA (L. TA) muscles and (FIG. 5B) diaphragm (Dia) from Dup2 mice treated with either PPMO-A1 or PPMO-Scr at the dose of 80 mg/kg, or saline (Sal) showing a dystrophin intensity across time points of 15, 30, and 60 days post-treatment in comparison to wild type (BI6) mice treated with saline. All intensity measurements are normalized to the BI6 cohort at each timepoint, and the mean (line)±range (shaded) is plotted for each group. Indicated significant differences, identified by Welch's ANOVA with Dunnett's T3 multiple comparison test, reflect comparisons based on the mean fiber intensity per tissue for all groups (n=3-6 per group). *p<0.05; **p<0.01; ***p<0.001; ****p<0.0001. Representative 1 mm2 regions of (FIG. 5C) left TA and (FIG. 5D) diaphragm sections with laminin in green and dystrophin in red showing localization and intensity of dystrophin expression. Color-coded heatmaps of each region display the normalized dystrophin intensity at the perimeter of each muscle fiber. Color key in the bottom corner shows color to percent intensity conversion for dystrophin intensity heatmaps, where 100% corresponds to the median fiber intensity in BI6 muscle.

FIG. 6A-D shows dystrophin induction by PPMO-A1 in the heart. (FIG. 6A) Example image of whole heart section showing laminin immunofluorescence (IF) and three-square regions of interest (ROIs) used in heart expression analysis. ROIs measured 0.5 mm on each side and were sampled from areas of cross-sectional cardiomyocyte orientation. (FIG. 6B) Quantification of dystrophin-positive myocytes in hearts at 15, 30, and 60 days after PPMO-A1, PPMO-Scr, or saline (Sal) treatment in Dup2 mice, with individual points representing each mouse. Wild type (BI6) mice were used as control groups. The data shown as mean±SEM (n=3-6 per group). All statistically significant differences identified by Welch's ANOVA with Dunnett's T3 multiple comparison test. *p<0.05; ****p<0.0001. (FIG. 6C) Cardiomyocyte dystrophin intensity in Dup2 treated with PPMO-A1, scrambled PPMO (PPMO-Scr), or saline (Sal) at 15, 30, and 60 days post-injection. All intensity measurements are normalized to the BI6 cohort at each timepoint, and the mean (line)±range (shaded) is plotted for each group. Indicated significant differences reflect comparisons based on the mean myocyte intensity per tissue for all groups (n=3-6 per group). All statistically significant differences identified by Welch's ANOVA with Dunnett's T3 multiple comparison test. **p<0.01; ****p<0.0001. (FIG. 6D) Representative 0.5×0.5 mm heart ROIs with laminin in green and dystrophin in red showing localization and intensity of dystrophin expression at the 15-day timepoint. Color-coded heatmaps of each ROI display the percent of the myocyte perimeter with dystrophin-positive pixels and the normalized dystrophin intensity at the perimeter of each myocyte. Color keys below each column shows the color to percent conversion for each type of heatmap. Myocytes with ≥70% dystrophin-positive perimeter are considered overall positive for dystrophin, and 100% on the intensity scale corresponds to the median myocyte intensity in BI6 hearts.

FIG. 7A-B shows restoration of dystrophin detected by western blot 15, 30, and 60 days following PPMO-A1 injection. (FIG. 7A) Western blot images of left tibialis anterior (L. TA) muscle showing dystrophin expression in Dup2 PPMO-A1 treated mice at 15, 30 and 60 days. Three samples from wild type (BI6) mice were used as a control pool and loaded twice on each gel for protein quantification assessment. Arrows show 460 kDa band from HiMark Pre-stained Protein Standard. (FIG. 7B) The dystrophin level (%) in Dup2 mice treated with PPMO-A1, scrambled PPMO (PPMO-Scr), and saline (Sal) was normalized to the mean in corresponding BI6 tissue. Data reported as mean±SEM for each group and time point. Statistical analysis was performed using Welch's ANOVA with Dunnett's T3 multiple comparison; *p<0.05; **p<0.01; ***p<0.001; ****p<0.0001.

FIG. 8 shows representative images of PPMO-induced DMD exon 2 skipping. Exon 2 exclusion in Dup2 mice (n=5-6) treated for 7 days with a dose escalation of PPMO-A1 (20, 40 and 80 mg/kg), PPMO-Scr (80 mg/kg) or saline (Dup2-Sal), and C57BI/6 (BI6-Sal) (n=5) injected with saline as a control. Right side of gastrocnemius (R. Gas), tibialis anterior (R. TA), diaphragm (Dia) and heart in all groups was used in RT-PCR analysis. Dup2, wild type (WT) and Del2 mRNA transcripts are represented as 340 bp, 278 bp and 216 bp bands, respectively. † is RT(−) and ‡ is PCR(−) negative controls, in which water was used as a template for RT and PCR steps to ensure that the tested samples were free of DNA contamination and non-specific amplification. Ladder, 1 kb Plus DNA ladder.

FIG. 9 shows representative images of PPMO-induced DMD exon 2 skipping. Exon 2 exclusion in Dup2 mice at 15, 30 and 60 days post injection with (n=5-6) treated with PPMO-A1 (80 mg/kg), PPMO-Scr (80 mg/kg) or saline (Dup2-Sal), and C57BI/6 (BI6-Sal) (n=5) injected with saline as a control. Right side of gastrocnemius (R. Gas), tibialis anterior (R. TA), diaphragm (Dia) and heart in all groups was used in RT-PCR analysis. Dup2, wild type (WT) and Del2 mRNA transcripts are represented as 340 bp, 278 bp and 216 bp bands, respectively. † is RT(−) and ‡ is PCR(−) negative controls, in which water was used as a template for RT and PCR steps to ensure that the tested samples were free of DNA contamination and non-specific amplification. Ladder, 1 kb Plus DNA ladder.

FIG. 10 shows Western blot images of dystrophin in heart (top) and diaphragm (Dia) (bottom) muscles at 15, 30 and 60 days in Dup2 mice treated with either PPMO-A1 or PPMO-Scr at the dose of 80 mg/kg and saline. Three wild type (BI6) mice were combined and loaded as a control pool twice on each gel for protein quantification assessment. *There were only three heart samples in PPMO-A1 treated cohort at 60 days, which could be used for analysis; **there were only three Dia samples in PPMO-Scr treated cohort at 30 days, which could be used for analysis.

FIG. 11A-D shows central nucleation in muscle after treatment with PPMO-A1. The percents centrally nucleated (CN) fibers in Dup2 (FIG. 11A) left TA muscles and (FIG. 11B) diaphragms are shown as mean±SEM for each treatment group and time point, with individual data points showing the percent CN fibers for each mouse. Statistically significant differences identified by ordinary one-way ANOVA with Sidak's multiple comparison test. *p<0.05; **p<0.01; ***p<0.001; ****p<0.0001. Representative 0.5×0.5 mm regions of (FIG. 11C) left TA and (FIG. 11D) diaphragm sections with laminin in green and DAPI in blue show peripheral and central nuclear localization. Color-coded fiber maps of each region display the automated categorization of each fiber as centrally nucleated (CN) or not (Non-CN) based on detected DAPI signal location.

FIG. 12A-C shows effects of PPMO-A1 treatment on muscle fiber size. Histograms of fiber minimum Feret's diameter in (FIG. 12A) left TA muscles and (FIG. 12B) diaphragms show fiber size in Dup2 mice treated with PPMO-A1, scrambled PPMO (PPMO-Scr), or saline (Sal) in comparison to wild type (BI6) muscle. (FIG. 12C) shows summary quantification of the fiber size as minimum Feret's diameter mean±SEM for each treatment group and time point, with individual data points showing the mean for each mouse. Indicated significant differences reflect comparisons based on the mean minimum Feret's diameter per tissue for all groups (n=3-6 per group). Statistically significant differences identified by ordinary one-way ANOVA with Sidak's multiple comparison test; *p<0.05; **p<0.01.

FIG. 13 shows the results of quantification of exon skipping at 7 days post-injection in the right tibialis anterior (RTA), right gastroctnemius (RGas), right quadriceps (RQuad), heart, and diaphragm (Dia). It represents the animal-level data summarized in the graph in FIG. 2, and shows the results from ascending doses of PPMO (20 mg/kg, 40 mg/kg, and 80 mg/kg). The quantification was performed via analysis of the amplicons in FIG. 8, using the ImageJ analysis software.

FIG. 14 shows the results of quantification of exon skipping at 15, 30, and 60 days post-injection in the right tibialis anterior (RTA), right gastrocnemius (RGas), right quadriceps (RQuad), heart, and diaphragm (Dia). PPMO was delivered at the dose of 80 mg/kg. It represents the animal-level data graphed in FIG. 3. The quantification was performed via quantification of the amplicons demonstrated in FIG. 8, using the ImageJ analysis software.

FIG. 15A-B shows the results of dystrophin expression analysis after treatment of Dup2 mice with PPMO-A1. FIG. 15A shows the Western blot expression of dystrophin from muscle lysates in left tibialis anterior (L.TA), diaphragm, and heart at 15, 30, and 60 days after treatment with PPMO-A1. Western blots for dystrophin were performed on muscle lysates that were loaded to match total protein content as determined by a DC assay. Each blot included 3 test samples and a 6-point standard curve made by diluting pooled wild type tissue samples dystrophin from six C57BI/6 mice in dystrophin-null lysate from age-matched Dup2Del18-41 mice, a spontaneous mouse model that developed from the Dup2 mouse model and a spontaneous deletion of exons 18-41 of the dmd gene. Dup2Del18-41 mice do not express any dystrophin compared to the Dup2 mice that express ˜5 of dystrophin. FIG. 15B shows quantitative analysis of percent dystrophin expression in the muscle lysates in left tibialis anterior (L.TA), diaphragm, and heart at 15, 30, and 60 days after treatment with saline (control), PPMO-A1 (PPMO-A), or PPMO-Scr (control). Dystrophin bands were analyzed using Image Lab software by quantifying the area under the curve of individual mouse samples against a linear regression line fit to the standard curve bands on each blot (R²□0.927 for each standard curve). Samples 1, 5 and 6 from the 60-day group were found to have a spontaneous deletion of Dmd exons 18-41 (Dup2Del18-41), and were thus excluded from quantification. All blot images representing PPMO-A1-treated samples (i.e., designated PPMO-A in the figure) are shown. Statistical results reflect Welch's ANOVA with Dunnett's T3 multiple comparison test; *p<0.05; **p<0.01; ***p<0.001.

### DETAILED DESCRIPTION

Duchenne muscular dystrophy (DMD) is caused by mutations that disrupt the reading frame of the DMD gene, leading to lack of dystrophin protein. Exon skipping therapies are designed to exclude a specific exon from the mRNA and restore the reading frame. Two such antisense phosphorodiamidate morpholino oligomers (PMO) have been approved for therapeutic use. These target exon 51 or exon 53 to result in expression of an internally-deleted but functional dystrophin protein.

In contrast, skipping of one copy of an exon in a patient with a single exon duplication may restore an entirely wild-type DMD transcript and dystrophin protein. In the case of exon 2 duplications—the most common duplication mutation in DMD—there is a wide therapeutic window: complete exon exclusion will result in expression from a downstream internal ribosome entry site (IRES) of an N-deleted protein with nearly normal function, as patients who express the DMD gene in this fashion are able to walk into the eight decade.

The disclosure provides products and methods for preventing, delaying the progression of, and/or treating subjects with one or more 5′ mutations of the DMD gene. As used herein, a “5′ mutation of the DMD gene” is a mutation within or affecting exon 1, 2, 3, 4, and or 5 of the DMD gene. In some aspects, the 5′ mutation is a DMD exon 2 duplication.

The disclosure provides oligonucleotides and antisense oligonucleotide constructs (i.e., peptide-conjugated oligonucleotides). In some aspects, the peptide-conjugated oligonucleotides comprise cell-penetrating peptides. Thus, in some aspects the disclosure provides peptide-linked phosphorodiamidate morpholino oligonucleotides (PMO) and cell penetrating peptide-conjugated PMOs (PPMOs) for treating, ameliorating, or preventing a muscular dystrophy resulting from a 5′mutation of the DMD gene. In some aspects, the oligonucleotide or the antisense oligonucleotide construct targets the exon 2 splice acceptor site where skipping of exon 2 is the primary endpoint. In some aspects, such antisense oligonucleotide constructs are PMO or PPMO. Thus, the disclosure provides PMO or PPMO for antisense-mediated exon-skipping to skip frame-disrupting exon(s) and allow functional dystrophin protein expression by restoring the reading frame. Antisense PMO have been well studied as promising tools with potential to block ribonucleic acid transcription (Summerton, J; Weller, D (1997); Antisense & Nucleic Acid Drug Development 7 (3): 187-95), and as such have potential value as therapeutics whose purpose is to control protein expression.

The term “polynucleotide,” as used herein, includes and/or is synonymous with “nucleic acid,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

As used herein, the terms “oligonucleotide” or “antisense oligonucleotide” or “oligomer” or “antisense oligomer” are used interchangeably to mean small molecules, usually, but not limited to, from about 8-50 nucleotides in length that bind via Watson-Crick base pairing to enhance or repress the expression of target RNA. Oligonucleotides are short single-stranded segments of DNA that upon cellular internalization can selectively inhibit the expression of a single protein. For antisense applications oligonucleotides interact and form a duplex with the mRNA or the pre-mRNA and inhibit its translation or processing, consequently inhibiting protein biosynthesis. The oligonucleotide is designed to block or inhibit translation of mRNA or to inhibit natural pre-mRNA splice processing, or induce degradation of targeted mRNAs, and may be said to be “directed to” or “targeted against” a target sequence with which it hybridizes. In certain embodiments, the target sequence is a region surrounding or including an AUG start codon of an mRNA, a 3′ or 5′ splice site of a pre-processed mRNA, or a branch point. The target sequence may be within an exon or within an intron or a combination thereof. The target sequence for a splice site may include an mRNA sequence having its 5′ end at 1 to about 25 base pairs downstream of a normal splice acceptor junction in a preprocessed mRNA. An exemplary target sequence for a splice site is any region of a preprocessed mRNA that includes a splice site or is contained entirely within an exon coding sequence or spans a splice acceptor or donor site. An oligomer is more generally said to be “targeted against” a biologically relevant target such as DMD gene pre-mRNA encoding the dystrophin protein, when it is targeted against the nucleic acid of the target in the manner described above. Exemplary targeting sequences include the nucleotide sequence set out in SEQ ID NO: 1 or 2.

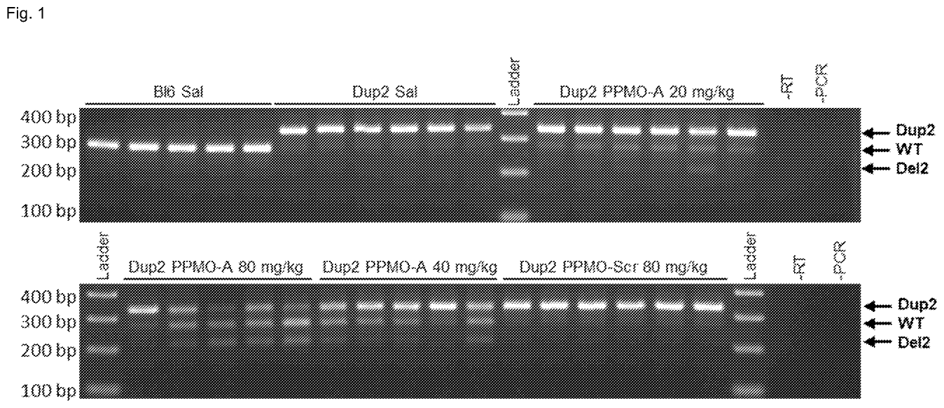

The disclosure provides various nucleotide sequences as set out in Table 1 below. In some aspects, the disclosure includes an oligonucleotide comprising a nucleotide sequence, or a variant thereof of the sequence forth in SEQ ID NO: 1 or 2. In some aspects, the disclosure includes an antisense oligonucleotide construct comprising the sequence set forth in SEQ ID NO: 1 or 2 conjugated to a peptide moiety. In the experiments set out in the drawings of FIG. 1-12C, “PPMO-A” shown in the drawings is “PPMO-A1”. The oligonucleotide comprising SEQ ID NO: 1 or 2, or a variant thereof, or the oligonucleotide construct comprising the oligonucleotide designated “PPMO-A1” or “PPMO-A2”, is designed to target exon 2 in treating muscular dystrophies resulting from 5′ mutations in the DMD gene. PPMO-scrambled (“PPMO-Scr”) is a control sequence. SEQ ID NO: 4 represents target sequence for PPMO-A1 on the DMD gene. SEQ ID NOs: 5 and 6 represent reverse and complementary sequences of PPMO-A1, respectively.

TABLE 1
Sequences of the Disclosure.

| | | | SEQ |
|---|---|---|---|
| | | | ID |
| No. | Name | Sequence | NO: |
| 1 | PPMO-A1 | GTTTTCTTTTGAACATCTTCTCTTTCAT | 1 |
| | | CTA | |
| 2 | PPMO-A2 | TAGATGAAAGAGAAGATGTTCAAAAGAA | 2 |
| | | AAC | |
| 3 | PPMO-Scr | GTATCTCTATTATTCATCGTCTTATTCT | 3 |
| | (control) | TC | |
| 4 | Target | TAGATGAAAGAGAAGATGTTCAAAAGAA | 4 |
| | sequence | AAC | |
| | for | | |
| | PPMO-A | | |
| 5 | Reverse A | ATCTACTTTCTCTTCTACAAGTTTTCTT | 5 |
| | | TTG | |
| 6 | Complement | CAAAAGAAAACTTGTAGAAGAGAAAGTA | 6 |
| | A | GAT | |

The disclosure provides an oligonucleotide that comprises, consists essentially of, or consists of a nucleotide sequence set forth in SEQ ID NO: 1 or 2. The disclosure also includes variants of these oligonucleotides comprising 80%, 81%, 82%, 83%, 84%, 85%, 86%, 87%, 88%, 89%, 90%, 91%, 92%, 93%, 94%, 95%, 96%, 97%, 98%, or 99% sequence identity or sequence homology to SEQ ID NO: 1 or 2, and/or variants that differ from these sequences by about 1, 2, 3, 4, 5, 6, 7, 8, 9, or 10 nucleotides, preferably those variants that modulate dystrophin expression in a cell. Also included are oligonucleotides comprising the sequence set forth in SEQ ID NO: 1 or 2, which comprise a suitable number of cationic or other modified linkages, as described herein, e.g., up to about 1 per every 2-5 uncharged linkages, such as about 4-5 per every 10 uncharged linkages, and/or which comprise a peptide or a cell-penetrating transport peptide attached thereto, as described herein.

In some aspects, the disclosure includes an oligonucleotide comprising a nucleotide sequence that is complementary to a nucleotide sequence that has at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to a sequence set forth in any one of SEQ ID NOs: 1 and 2.

In some aspects, the disclosure includes an oligonucleotide comprising a nucleotide sequence that binds the target sequence of the DMD gene, i.e., SEQ ID NO: 4.

In some aspects, the disclosure includes an oligonucleotide comprising a nucleotide sequence set forth in SEQ ID NO: 5 or 6.

The term “polyribonucleotide,” as used herein, includes “ribonucleic acid,” “oligoribonucleotide,” and “ribonucleic acid molecule,” and generally means a polymer of RNA which can be single-stranded or double-stranded, synthesized or obtained (e.g., isolated and/or purified) from natural sources, which can contain natural, non-natural or altered nucleotides, such as morpholinos, and which can contain a natural, non-natural or altered internucleotide linkage, such as a phosphoroamidate linkage or a phosphorothioate linkage, instead of the phosphodiester found between the nucleotides of an unmodified oligonucleotide. It may be suitable in some instances, in an embodiment, for the nucleic acids to comprise one or more insertions, deletions, inversions, and/or substitutions.

The nucleic acid can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

In some aspects, an oligonucleotide as described herein is conjugated to a peptide or a cell-penetrating peptide (CPP) in order to enhance the delivery of the oligonucleotide to the cell of the subject. As used herein, the terms “peptide-conjugated oligonucleotides” or “peptide oligonucleotide conjugates” or “antisense oligonucleotide construct” are used interchangeably to mean molecular composites containing a nucleic acid or oligonucleotide moiety covalently linked to a polypeptide moiety. The antisense oligonucleotide construct serves many important roles as potential therapeutics and owing to their stability, can resist intracellular enzymes present in different cellular compartments.

In some aspects, such “antisense oligonucleotide construct” comprises a peptide-conjugated oligonucleotide or a cell-penetrating peptide-conjugated oligonucleotide. As used herein, the terms “cell penetrating peptide” or “CPP” are used interchangeably herein to mean short peptides that facilitate cellular intake and uptake of molecules ranging from nanosize particles to small chemical compounds to large fragments of DNA. In some aspects, the oligonucleotide is associated with the peptide or CPP either through chemical linkage via covalent bonds or through non-covalent interactions. In some aspects, CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake. In some aspects, CPPs refers to cationic cell penetrating peptides, also called transport peptides, carrier peptides, or peptide transduction domains. The disclosure includes the use of various peptides and cell-penetrating peptides, as described by Roberts et al. (Nature Reviews 19: 673-94, 2020), which is incorporated herein by reference in its entirety.

In some aspects, the peptide-conjugated oligonucleotides or CPP-conjugated oligonucleotides include, but are not limited to, phosphoramidate morpholino oligomers and phosphorodiamidate morpholino oligomers (PMO), phosphorothioate modified oligomers, 2′ O-methyl modified oligomers, peptide nucleic acid (PNA), locked nucleic acid (LNA), phosphorothioate oligomers, 2′ O-MOE modified oligomers, 2′-fluoro-modified oligomer, 2′O,4′C-ethylene-bridged nucleic acids (ENAs), tricyclo-DNAs, tricyclo-DNA phosphorothioate nucleotides, 2′-O-[2-(N-methylcarbamoyl)ethyl] modified oligomers, morpholino oligomers, peptide-conjugated phosphoramidate morpholino oligomers (PPMO), phosphorodiamidate morpholino oligomers having a phosphorous atom with (i) a covalent bonds to the nitrogen atom of a morpholino ring, and (ii) a second covalent bond to a (1,4-piperazin)-1-yl substituent or to a substituted (1,4-piperazin)-1-yl (PMOplus), and phosphorodiamidate morpholino oligomers having a phosphorus atom with (i) a covalent bond to the nitrogen atom of a morpholino ring and (ii) a second covalent bond to the ring nitrogen of a 4-aminopiperdin-1-yl (i.e., APN) or a derivative of 4-aminopiperdin-1-yl (PMO-X) chemistries, including combinations of any of the foregoing. In general, PNA and LNA chemistries can utilize shorter targeting sequences because of their relatively high target binding strength relative to PMO and 2′O-Me modified oligomers. Phosphorothioate and 2′O-Me-modified chemistries can be combined to generate a 2′O-Me-phosphorothioate backbone. See, e.g., PCT Publication Nos. WO2013/112053 and WO2009/008725, which are hereby incorporated by reference in their entireties.

In some aspects, therefore, the “antisense oligonucleotide construct” is s PMO or PPMO. In some aspects, the antisense oligonucleotide construct is PPMO-A1 or PPMO-A2. In some aspects, such construct comprises a sequence of subunits, each having a pyrimidine or purine nucleobase carried on a backbone subunit composed of a morpholino group, and where the backbone groups are linked by substantially uncharged phosphorodiamidate groups that allow the bases in the compound to hybridize to a target sequence, such as, for example, in DMD mRNA by Watson-Crick base pairing, to form an RNA:PMO heteroduplex within the target sequence. The PMO or PPMO may have substantially complete complementarity to the RNA target domain or near complementarity so that the degree of complementarity is in the range of about 80% to about 100%. PMO or PPMO are designed to block or inhibit translation of the mRNA containing the target sequence.

The term “target sequence” refers to a portion of the target RNA against which the antisense agent is directed and will hybridize by Watson-Crick base pairing of an essentially complementary or nearly complementary sequence. In some aspects, the target sequence comprises the exon 2 splice acceptor site of the DMD gene. In some aspects, the target sequence is set forth in SEQ ID NO: 4.

As used herein, the term “complementarity” or “complementary” means that a nucleic acid can form hydrogen bond(s) with another nucleic acid sequence by either traditional Watson-Crick or other non-traditional types. In reference to the polyribonucleotide molecules of the disclosure, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). In some specific aspects, the terms “complementary” and “complementarity” refer to polynucleotides (i.e., a sequence of nucleotides) related by the base-pairing rules. For example, the sequence “T-C-G-G-T,” is complementary to the sequence “A-G-C-C-A.” Complementarity may be “partial,” in which only some of the nucleic acids' bases are matched according to the base pairing rules. Or, there may be “complete” or “total” complementarity between the nucleic acids.

The term “knock down” refers to the inhibition or blocking of protein synthesis due to steric inhibition of the transcription process. In one aspect of the present invention, said knock down is a result of Watson-Crick pairing of an antisense PMO or PPMO to RNA encoding the dystrophin protein or a mutant thereof, which results in skipping of exon 2 from the mRNA. Thus, the PMO or PPMO of the disclosure skips DMD exon 2 and restores the reading frame of the DMD gene resulting in therapeutic transcripts lacking one or both copies of exon 2 which result in a functional dystrophin protein providing the prophylaxis or treatment of muscular dystrophy caused by the mutant dystrophin protein. The products and methods described herein are used for preventing disease, delaying the progression of disease, and/or treating muscular dystrophies in patients with one or more mutations of the DMD gene. In some aspects, the products and methods described herein are used for treating one or more mutations within, surrounding, or affecting the 5′ end of the DMD gene. In some aspects, the mutation is within, surrounding, or affecting any one or more of exons 1-5. In some aspects, the mutation is a mutation of exon 2 including, but not limited to, a duplication of exon 2. The products and methods induce skipping of exon 2 of the DMD gene to allow for the expression of a dystrophin protein with some N-deletion with normal or nearly normal function.

The disclosure also provides PMO or PPMO, compositions comprising the PMO or PPMO, and methods useful for treating 5′ mutations in the DMD gene in a subject. In some aspects, the PMO or PPMO of the disclosure induce skipping exon 2 of DMD by interfering with splice acceptor site of exon 2 of the DMD target gene, resulting in skipping of exon 2. Thus, as used herein, the disclosure provides PMOs or PPMOs and methods used to modulate the expression of the DMD gene by skipping exon 2.

The term “modulate,” as used herein means that the expression of the DMD gene, or level of RNA molecule or equivalent RNA molecules encoding the dystrophin protein is affected by the binding of the PMO or PPMO antisense oligomer.

In some aspects of the disclosure, a PMO or PPMO molecule with scrambled sequence or with mismatches is used as a control. In some aspects, such sequence is designated “PPMO-Scr” and comprises the oligonucleotide sequence set out in SEQ ID NO: 3.

The amount of time of exposure of the PMO or PPMO to the host cells, population of cells or subject should be sufficiently long to effect skipping of exon 2 or modulation of the expression of the DMD gene in the host cell, population of cells, or in the subject. The time for the desired effect varies with dosage, target, age and other factors known to those of skill in the art. Generally, the time of exposure of the PMO or the PPMO to the host cells, population of cells or subject should range from about 1 hour to about 60 days, from about 1 hour to about 30 days, from about 1 hour to about 15 days, from about 1 hour to about 14 days, from about 1 hour to about 13 days, from about 1 hour to about 12 days, from about 1 hour to about 11 days, from about 1 hour to about 10 days, from about 1 hour to about 9 days, from about 1 hour to about 8 days, from about 1 hour to about 7 days, from about 1 hour to about 6 days, from about 1 hour to about 5 days, from about 1 hour to about 4 days, from about 1 hour to about 3 days, from about 1 hour to about 48 hours, from about 1 hour to about 36 hours, from about 1 hour to about 24 hours, from about 1 hour to about 12 hours, from about 1 hour to about 11 hours, from about 1 hour to about 10 hours, from about 1 hour to about 9 hours, from about 1 hour to about 8 hours, from about 1 hour to about 7 hours, from about 1 hour to about 6 hours, from about 1 hour to about 5 hours, from about 1 hour to about 4 hours, from about 1 hour to about 3 hours, or from about 1 hour to about 2 hours.

In some aspects, the disclosure provides a composition or compositions comprising at least one or more of the PMO or PPMO as described herein. In various aspects, such compositions also comprise a pharmaceutically acceptable carrier. In various aspects, such compositions also comprise other ingredients, such as a diluent, excipients, and/or adjuvant. Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating the PMO or PPMO, or the compositions comprising same, in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The amount or dose of the PMO or PPMO of the disclosure that is administered should be sufficient to effectively target the cell, or population of cells in vivo, such that the modulation of the expression of the target gene of interest can be detected, in the subject over a reasonable time frame. The dose will be determined by the efficacy of the particular PMO or PPMO formulation and the location of the muscle cells to be targeted in the subject, as well as the body weight and, in some aspects, the age of the subject to be treated. The dose of the PMO or the PPMO to be administered in methods of the disclosure will also vary depending, for example, on the particular PMO or PPMO, the mode of administration, the treatment goal, the individual, and the cell type(s) being targeted, and may be determined by methods standard in the art.

The dose of the PMO or PPMO of the disclosure also will be determined by the existence, nature and extent of any adverse side effects that might accompany the administration. Typically, an attending physician will decide the dosage of the PMO or PPMO with which to treat each individual subject, taking into consideration a variety of factors, such as age, body weight, general health, diet, sex, compound to be administered, route of administration, and the severity of the condition being treated. By way of example, and not intending to limit the invention, the dose of the PMO or PPMO can be about 0.001 to about 1000 mg/kg body weight of the subject being treated, from about 0.01 to about 100 mg/kg body weight, from about 0.1 mg/kg to about 100 mg/kg body weight, or from about 1.0 mg/kg to about 100 mg/kg body weight. In some aspects, the dose of PMO or PPMO administered is about 20 mg/kg to about 80 mg/kg body weight. In another embodiment, the dose of the PMO or the PPMO can be at a concentration from about 1 nM to about 10,000 nM, preferably from about 1 μM to about 50 μM.

In some aspects, the dose of PMO or PPMO administered is about 0.1 mg/kg about 1 mg/kg, about 10 mg/kg, about 100 mg/kg, or about 1000 mg/kg body weight,

In some aspects, the dose of PMO or PPMO administered is about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg body weight.

In some aspects, an initial dose is followed by a second greater dose. In some aspects, an initial dose is followed by a second same dose. In some aspects, an initial dose is followed by one or more lesser doses. In some aspects, an initial dose is followed by multiple doses which are the same or greater doses.

The disclosure thus provides compositions and methods of administering/delivering the PMO or PPMO which express antisense sequence that binds to key exon definition elements in the pre-mRNA, inhibiting the recognition of a specific exon by the spliceosome, leading to exclusion of the target exon from the mature RNA to a subject. In some aspects, the subject is a mammal. In some aspects, the mammal is a human.

The in vivo methods comprise the step of administering an effective dose, or effective multiple doses, of a composition comprising the PMO or PPMO to a subject (including a human subject) in need thereof. Thus, methods are provided of administering an effective dose (or doses, administered essentially simultaneously or doses given at intervals) of the PMO or PPMO described herein to a subject in need thereof. If the dose or doses is administered prior to development of a disorder/disease, the administration is prophylactic. If the dose or doses is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

The disclosure includes products and methods for preventing, delaying the progression of, and/or treating patients with one or more 5′ mutations of the DMD gene within, surrounding, or affecting exon 2 of the DMD gene. In some aspects, such mutation results in an exon 2 duplication.

In some embodiments, a method of the disclosure comprises the step of administering a PMO or PPMO construct that is an exon 2-targeting antisense oligomer. In some embodiments, the method is for treating a subject who suffers from or is at risk of suffering from a muscular dystrophy. In some aspects, the subject is also treated with an additional therapy. In some aspects, the additional therapy is a glucocorticoid. In some aspects, the subject is a human.

In some aspects, compositions and methods of the disclosure are used in treating, ameliorating, or preventing a disease, such as a muscular dystrophy (MD). In various aspects, such MD is Duchenne Muscular Dystrophy (DMD). DMD, an X-linked degenerative muscle disorder, is the most common severe childhood form of muscular dystrophy affecting around 1:5200 male births (Mendell et al., Ann Neurol 71, 304-313 (2012)). Symptoms of generalized muscle weakness first appear at ages 3-5 and progress into a loss of ambulation by age 13, with death typically occurring in the third decade of life due to cardiomyopathy or respiratory insufficiency (Passamano et al., Acta Myol 31, 121-125 (2012); Duchenne, The Pathology of Paralysis with Muscular Degeneration (Paralysie Myosclerotique), or Paralysis with Apparent Hypertrophy. Br Med J 2, 541-542 (1867)). DMD is caused by mutations that disrupt the open reading frame in the DMD gene, which encodes dystrophin (Juan-Mateu et al., PLOS One 10, e0135189 (2015)), a large (427 kDa) multifunctional protein that is localized at the subsarcolemmal region of myofibers, where it plays an important role in protecting the sarcolemma from mechanical damage caused by muscle contraction (Petrof et al., Proc Natl Acad Sci USA 90, 3710-3714 (1993)). In other various aspects, such MD is Becker Muscular Dystrophy (BMD). The presence of a partially functional dystrophin protein occurs with mutations that maintain an open reading frame (ORF), resulting in the milder allelic disorder BMD (Wein et al., Nature Medicine 20, 992-1000 (2014); Monaco, Trends Biochem Sci 14, 412-415 (1989)). BMD, like DMD, is a genetic disorder that gradually makes the body's muscles weaker and smaller. BMD affects the muscles of the hips, pelvis, thighs, and shoulders, as well as the heart, but is known to cause less severe problems than DMD. Because of the variety of in-frame mutations resulting in a variety of partially functional proteins, BMD has a broad phenotypic spectrum with, for example, loss of ambulation ranging from the late teenage years to late adulthood.

In families known to carry pathological DMD or BMD mutations, the methods of the disclosure, in various aspects, are methods of preventing disease and they are carried out before the onset of disease. In other various aspects, the methods of the disclosure are carried out after diagnosis and, therefore, are methods of treating or ameliorating disease.

Molecular, biochemical, histological, and functional outcome measures demonstrate the therapeutic efficacy of the methods. Outcome measures are described, for example, in Chapters 32, 35 and 43 of Dyck and Thomas, Peripheral Neuropathy, Elsevier Saunders, Philadelphia, PA, 4^{th} Edition, Volume 1 (2005) and in Burgess et al., Methods Mol. Biol., 602: 347-393 (2010). Outcome measures include, but are not limited to, one or more of the exclusion of the target exon from the mature RNA, reduction or elimination of mutant DMD mRNA or protein in affected tissues, and the expression of a functional form of dystrophin. The expression of functional dystrophin in the cell is detected by measuring the dystrophin protein level by methods known in the art including, but not limited to, Western blot, immunofluorescence, or immunohistochemistry in muscle biopsied before and after administration of the PMO or PPMO, or the composition to determine the improvement.

In some aspects, the level of functional dystrophin gene expression or protein expression in a cell of the subject is increased after administration of the PMO or PPMO as compared to the level of functional dystrophin gene expression or protein expression before administration of the PMO or PPMO. In some aspects, expression of a functional form of dystrophin is increased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, improved muscle strength, improved muscle function, and/or improved mobility and stamina show an improvement by at least about 2%, at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%.

Other outcome measures include measuring the level of serum creatinine kinase (CK) in the subject before and after treatment. Increased CK levels are a hallmark of muscle damage. In Duchenne patients, CK levels are significantly increased above the normal range (10 to 100 times the normal level since birth). When elevated CK levels are found in a blood sample, it usually means muscle is being disintegrated by some abnormal process, such as a muscular dystrophy or inflammation. Thus, a positive therapeutic outcome for treatment with the methods of the disclosure is a reduction in the level of serum creatinine kinase after administration of the PMO or PPMO as compared to the level of serum creatinine kinase before administration of the PMO or PPMO, or the composition.

Other outcome measure include measuring to determine if there is improved muscle strength, improved muscle function, improved mobility, improved stamina, or a combination of two or more thereof in the subject after treatment. Such outcome measures are important in determining muscular dystrophy progression in the subject and are measured by various tests known in the art. Some of these tests include, but are not limited to, the six minute walk test, time to rise test, ascend 4 steps test, ascend and descend 4 steps test, North Star Ambulatory Assessment (NSAA) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

Combination therapies are also contemplated by the disclosure. Combination as used herein includes both simultaneous treatment and sequential treatments. Combinations of methods described herein with standard medical treatments and supportive care are specifically contemplated, as are combinations with therapies, such as glucocorticoids. All types of glucocorticoids are included for use in the combination therapies disclosed herein. Such glucocorticoids include, but are not limited to, prednisone, prednisolone, dexamethasone, deflazacort, beclomethasone, betamethasone, budesonide, cortisone, hydrocortisone, methylprednisolone, and triamcinolone.

Administration of an effective dose of a PMO, PPMO, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration of the PMO or PPMO of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of the PMO or PPMO may be accomplished by using any physical method that will transport the PMO or PPMO into a target cell of a subject. Administration includes, but is not limited to, injection into muscle, the bloodstream and/or directly into the nervous system or liver. Simply resuspending a PMO or PPMO in phosphate buffered saline has been demonstrated to be sufficient to provide a vehicle useful for muscle tissue expression, and there are no known restrictions on the carriers or other components that can be co-administered with the PMO or PPMO (although compositions that degrade DNA should be avoided). Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The PMO or PPMO can be used with any pharmaceutically acceptable carrier for ease of administration and handling.

A dispersion of PMO or PPMO can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating PMO or PPMO in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides a kit comprising a PMO, PPMO, or composition of the disclosure or produced according to a process of the disclosure. In the context of the disclosure, the term “kit” means two or more components, one of which corresponds to a PMO, PPMO, or composition of the disclosure, and the other which corresponds to a container, recipient, instructions, or otherwise. A kit, therefore, in various aspects, is a set of products that are sufficient to achieve a certain goal, which can be marketed as a single unit.

The kit may comprise one or more recipients (such as vials, ampoules, containers, syringes, bottles, bags) of any appropriate shape, size and material containing the PMO, PPMO, or composition of the disclosure in an appropriate dosage for administration (see above). The kit may additionally contain directions or instructions for use (e.g. in the form of a leaflet or instruction manual), means for administering the PMO, PPMO, or composition, such as a syringe, pump, infuser or the like, means for reconstituting the PMO, PPMO, or composition and/or means for diluting the PMO, PPMO, or composition.

In one embodiment, such a kit includes the PMO or PPMO, or the composition in a diluent packaged in a container such as a sealed bottle or vessel, with a label affixed to the container or included in the package that describes use of the PMO or PPMO, or the composition. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

The disclosure also provides kits for a single dose of administration unit or for multiple doses of the PMO or PPMO, or the composition. In some aspects, the disclosure provides kits containing single-chambered and multi-chambered pre-filled syringes.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

Unless otherwise indicated, the term “at least” preceding a series of elements is to be understood to refer to every element in the series. Those skilled in the art will recognize, or be able to ascertain using no more than routine experimentation, many equivalents to the specific aspects of the disclosure described herein. Such equivalents are intended to be encompassed by the disclosure.

The term “and/or” wherever used herein includes the meaning of “and”, “or” and “all or any other combination of the elements connected by said term.”

The term “about” or “approximately” as used herein means within 20%, preferably within 10%, and more preferably within 5% of a given value or range. It includes, however, also the concrete number, e.g., about 10 includes 10. Recitation of ranges of values herein are merely intended to serve as a shorthand method for referring individually to each separate value falling within the range and each endpoint, unless otherwise indicated herein, and each separate value and endpoint is incorporated into the specification as if it were individually recited herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular aspects only, and is not intended to limit the scope of the subject matter of the disclosure, which is defined solely by the claims.

All methods described herein are performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure.

### EXAMPLES

Aspects and aspects of the disclosure are illustrated by the following examples.

### Example 1

### Materials and Methods

### Animal Studies and Injections

Dup2 (12-week-old), the generation of which was previously described (Vulin et al., Neuromuscul Disord 25, 827-834, doi:10.1016/j.nmd.2015.08.005 (2015)), and age-matched C57BI/6 (BI6) mice (The Jackson Laboratory; stock #000664) were used in all experiments. All animal studies were performed according to the guidelines and approval of the Institutional Animal Care and Use Committee of the Abigail Wexner Research Institute at Nationwide Children's Hospital. All animal studies were performed in a double-blinded manner.

For dose-escalation studies, PPMO-A or PPMO-Scr were reconstituted in saline and injected into the tail vein of 12 week-old Dup2 mice (n=5-7) at final doses of 20, 40 and 80 mg/kg. Age-matched C57BI/6 mice (n=6) were also injected with saline, sacrificed at the same time-points and used as controls. Mice were sacrificed using a lethal dose of ketamine/xylazine (KX) cocktail (NCH pharmacy) seven days post PPMO injection for tissue collection. Tibialis anterior (TA), gastrocnemius (Gast), quadriceps (Quad), diaphragm (Dia), and heart were snap-frozen in liquid nitrogen-cooled isopentane and stored at −80° ° C. until use.

For a time-course study, 12-week-old Dup2 mice (n=4-7) were injected at the dose of 80 mg/kg and sacrificed 15-, 30- and 60-days post either PPMO-A1, PPMO-Scr or saline administration using a lethal dose of ketamine/xylazine (KX) cocktail. Age-matched C57BI/6 mice (n=6-7) were also injected with saline, sacrificed at the same time-points and used as controls. The same tissue as described for the dose-escalation study were collected and stored at −80° ° C. until use.

### Production of PPMO Compounds

The PPMO-A1 and PPMO-Scr peptide conjugates were synthesized and purified to about greater than 90% purity at Sarepta Therapeutics, Inc. PPMO-A1 comprising the 31-nt oligonucleotide (GTTTTCTTTTGAACATCTTCTCTTTCATCTA; SEQ ID NO: 1) was designed to target exon 2 splice acceptor site of DMD gene. Due to 100% homology of the target region, the PPMO-A1 sequence is applicable to be used in both mouse and human DMD gene correction studies. PPMO-Scr sequence comprising the oligonucleotide (GTATCTCTATTATTCATCGTCTTATTCTTC; SEQ ID NO: 3) was designed as an antisense 30-nt long control for PPMO-A1 using online GenScript Bioinformatics software (https:-forward slash—forward slash-www.genscript.com-forward slash-tools-forward slash-create-scrambled sequence). Both PPMO-A1 and PPMO-Scr compounds were dissolved in sterile saline and their concentrations were measured in Sarepta's Therapeutics facility prior overnight shipping to a laboratory at Nationwide Children's Hospital (NCH; Columbus, OH) for further experiments. Compounds were stored at 4° C. and used during 30-45 days after resuspension.

### RNA Extraction and Exon 2 Skipping Analysis

Total RNA was extracted from frozen skeletal muscles, diaphragm, and heart through standard TRIzol/chloroform extraction (Life Technologies, cat #15596018, Carlsbad, CA, USA; Fisher Bioreagents, cat #C297-4, Hampton, NH) and then purified using RNA Clean & Concentrator-25 (Zymo Research, cat #R1018, Tustin, CA) according to manufacturer's instruction. The reverse transcription (RT) was performed on 1000 ng of total RNA with RevertAid RT Kit and random hexamer primers (Thermo Scientific, cat #K1691, Waltham, MA) according to manufacturer's protocol. cDNA was amplified via PCR (Thermo Fisher, cat #K0171, Waltham, MA) using primers specific to the DMD 5′UTR and the exon 3-4 junction.

TABLE 2
Primer Sequences.

| Name | Sequence | SEQ ID NO: |
|---|---|---|
| RT-dmd- | TACCTAAGCCTCCTGGAGCA | 7 |
| mice-1fw | | |
| RT-dmd- | CTTTTGGCAGTTTTTGCCCT | 8 |
| mice-1rv | GTA | |

Digital images of DNA bands on gels stained with ethidium bromide as an intercalating agent were quantified using ImageJ software (NIH, Version 1.46r, USA) to determine relative amounts of different amplicons.

### Protein Extraction and Western Blotting

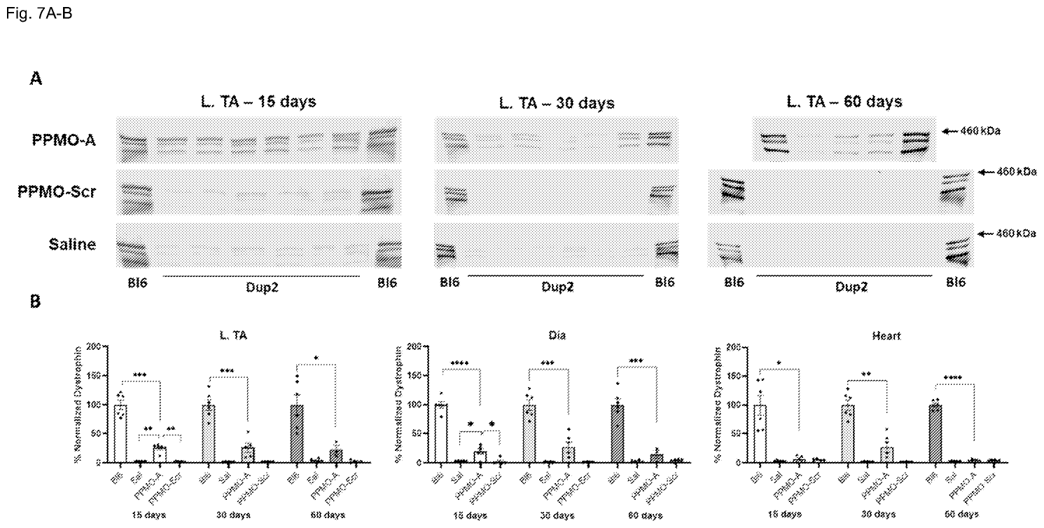

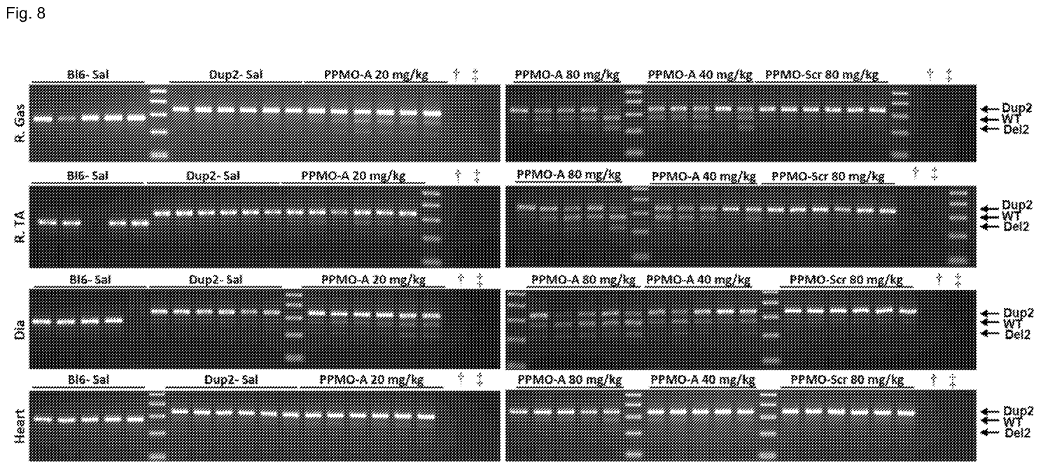

Mouse tissue lysates were prepared by mechanically disrupting tissue in a Tissuelyser II (Qiagen) in home-made lysis buffer comprising 50 mM Tris-HCl, 150 mM NaCl, 1% NP-40, digitonin (Sigma, cat #D141-500MG) and protease inhibitor cocktail (Complete mini, EDTA-free protease inhibitor cocktail tablet; cat #NC0962311, Sigma Aldrich). The lysate was subjected to centrifugation at 14,000 g for 20 min at 4° C. and supernatants were collected for analysis. The total proteins were quantified using a BCA protein assay kit (Pierce, cat #PI23227, Thermo Scientific, USA) following manufacturer's protocol. The supernatant was mixed with a 4× Laemmli sample buffer and boiled 5 min at 90-100° C. and 50 μg of total protein was run on a precast 3-8% Tris-Acetate gel (NuPage, cat #EA0378BOX, Invitrogen) for 1 h at 80 V then 4 h at 120 V (4° C.). Samples from C57BI/6 mice (pool of 3) were run as controls for dystrophin quantification. Proteins from gels were transferred on a P 0.45 μm PVDF membrane (Cytiva Life Sciences™ Amersham™ Hybond™, cat #10600023) at a constant 55 mA overnight with slow stirring at 4° C. HiMark Pre-stained protein standard (Invitrogen, cat #LC5699) and precision plus protein dual color standards (BioRad, cat #1610394) were used to determine the size of proteins of interest during gel running and protein transferring. For normalization, blots were incubated in REVERT™ Total Protein Stain (Li-cor, Cat #92611021, Li-Cor, USA) and scanned in the 700 channel on an Odyssey CLx Imaging System (Li-cor). To detect the expression of dystrophin, dystrophin anti-rabbit polyclonal antibody (Abcam, cat #ab15277) was used at 1:200 dilution Odyssey Buffer (Li-cor, cat #927-40000) with 0.1% Tween-20. After 2 h incubation at RT, membrane was washed (4×5 min with 0.1% Tween in PBS) and exposed to the secondary antibody, anti-rabbit IgG (H+L) IRDye 800CW conjugate (Li-cor, cat #926-32211) for 60 min at RT at 1:5000 dilution in Odyssey blocking buffer. Blots were washed 5×5 min with 0.1% Tween in PBS, followed by 1×5 min wash in PBS. Blots were immediately immersed in ddH₂O. Blots were scanned in the 800 channel on LI-COR Odyssey CLx. Total protein and dystrophin signal were analyzed using Li-cor Image Studio Lite software (Li-cor, USA). First, total protein signal in each lane was normalized to the highest lane's signal to produce the lane normalization factor (LNF) for quantification of protein. Next, dystrophin signal (two top bands) in each lane (e.g., see FIGS. 7A-B and 10) was normalized using the LNF. Finally, the normalized dystrophin signal from C57BI/6 control animals was averaged and test samples were normalised to this average to give final dystrohpin quantification.

### Immunohistochemistry and Microscopy

Frozen muscles were cut at 10 microns air-dried, permeabilized, blocked and co-stained 1:400 in rabbit monoclonal anti-dystrophin antibody (Abcam, cat #ab218198) and anti-laminin (R&D System, cat #MAB4656) for two hours at room temperature. Slides were washed 4×5 min, incubated in the appropriate Alexa Fluor 488 (Jackson Labs, cat #712-546-153) or 568 (Invitrogen, cat #A-21069) conjugated secondary antibodies (1:500 dilution) for 1 h and washed 3×5 minutes before cover slips were affixed with Vectashield Hard Set anti-fade mounting medium with DAPI (Vector Laboratories, cat #H-1500).

Whole section images of skeletal muscles and hearts were collected on a fully motorized Nikon Ti2-E inverted microscope with Plan Apochromat Lambda objectives and a Hamamatsu ORCA Fusion camera within 6 h of completion of staining. Skeletal muscles were imaged using 10× magnification at a resolution of 0.64 μm/pixel and hearts were imaged using 20× magnification at a resolution of 0.32 μm/pixel.

### Microscope Image Analysis

Analysis was carried out in Nikon NIS-Elements AR (Advanced Research) (Nikon) software using the General Analysis 3 software module and a custom analysis workflow developed for mouse tissue. Skeletal muscles were analyzed as whole tissue sections, using thresholds for dystrophin-positive and laminin-positive pixels that were empirically derived from the intensity profiles of both signals in untreated Dup2 tissue sections. Dystrophin-positive fibers were quantified by identifying all individual muscle fibers using laminin-positive boundaries, measuring the total length of dystrophin-positive segments around each muscle fiber, and normalizing it to the total length of the laminin-positive segment around the muscle fiber perimeter. The criterion for identifying a muscle fiber as overall positive for dystrophin was set at 70% or more of the perimeter. Fiber dystrophin intensity was analyzed by measuring the mean intensity of all pixels in the dystrophin channel within a 5 μm-thick boundary region around each muscle fiber.

Muscle fiber size was measured in terms of area and minimum Feret's diameter automatically as a part of the dystrophin analysis. In a separate analysis, centrally nucleated (CN) fibers were identified as those having at least one DAPI nucleus overlapping with the interior region of the fiber that was eroded in from the laminin-positive sarcolemma by 5 μm, and the % centrally nucleated fibers was calculated for each tissue.

Hearts were analyzed as 3 square regions of interest (ROIs) sampled from the transverse myocyte regions of each heart section, with each ROI measuring 0.5×0.5 mm. The ROI sampling was performed using the laminin channel by an operator blinded to the dystrophin channel. The analysis workflow used to quantify dystrophin-positive myocytes and myocyte dystrophin intensity in heart ROIs followed the same approach as the skeletal muscle analysis described above. Cardiac myocytes touching the edges of the ROI field were excluded from analysis. Quantification results from the 3 ROIs for each heart were pooled to produce a single result for dystrophin-positive myocytes and myocyte dystrophin intensities for each heart.

### Statistical Analysis

All results are presented as mean value±SEM. Differences between groups within a given timepoint were assessed by one-way ANOVA with Sidak's multiple comparison test for groups with equal standard deviations, or Welch's ANOVA with Dunnett's T3 multiple comparison test for groups with unequal standard deviations. Individual comparisons were performed for PPMO-A1 versus each control group (C57BI/6, PPMO-Scr and saline). Dose escalation results were analyzed by linear regression. Significance was determined based on α=0.05.

### Example 2

### Single Systemic Delivery of PPMO-A1 Induced the DMD Exon 2 Exclusion in a Dose-Dependent Manner in Dup2 Mice

The efficacy of the novel PPMO-A1 and its PPMO-Scrambled (PPMO-Scr) control compounds to skip exon 2 in 12-week-old Dup2 males (n=5-7) was evaluated. Mice were intravenously injected with PPMO-A1 at the doses of 20, 40 and 80 mg/kg. Because no significant therapeutic effect was expected in Dup2 mice treated with PPMO-Scr control, those animals were dosed at 80 mg/kg. Dup2 and C57BI/6 mice injected with saline were also used as controls. The study design is summarized in Table 3.

TABLE 3
Dose-Escalation Study Design.

| | | | | Doses | In-Life Duration |
|---|---|---|---|---|---|
| Strain | Sex | Compounds | n | (mg/kg) | (days) |
| Dup2 | Male | Saline | 7 | N/A | 7 |
| Dup2 | Male | PPMO-A1 | 6 | 20 | 7 |
| Dup2 | Male | PPMO-A1 | 5 | 40 | 7 |
| Dup2 | Male | PPMO-A1 | 5 | 80 | 7 |
| Dup2 | Male | PPMO-Scr | 6 | 80 | 7 |
| C57Bl/6 | Male | Saline | 6 | N/A | 7 |

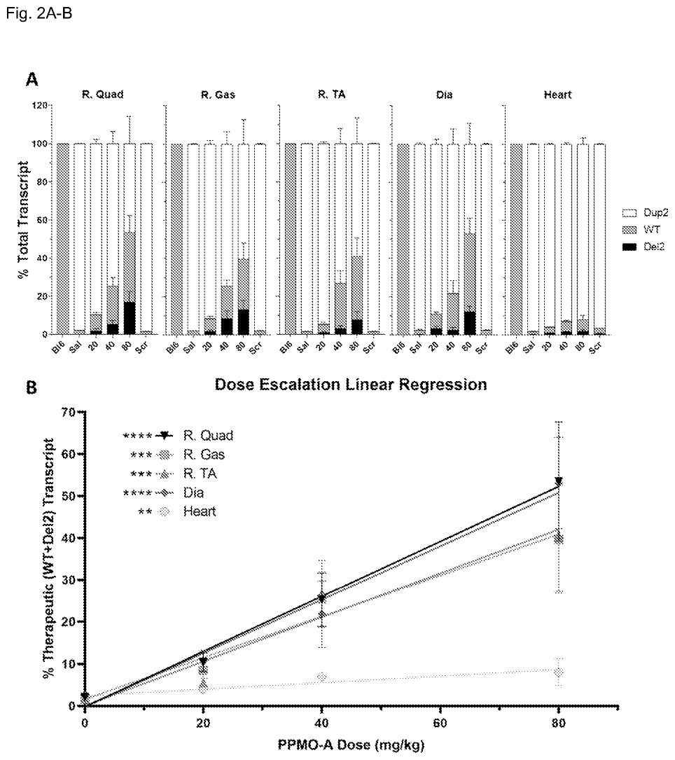

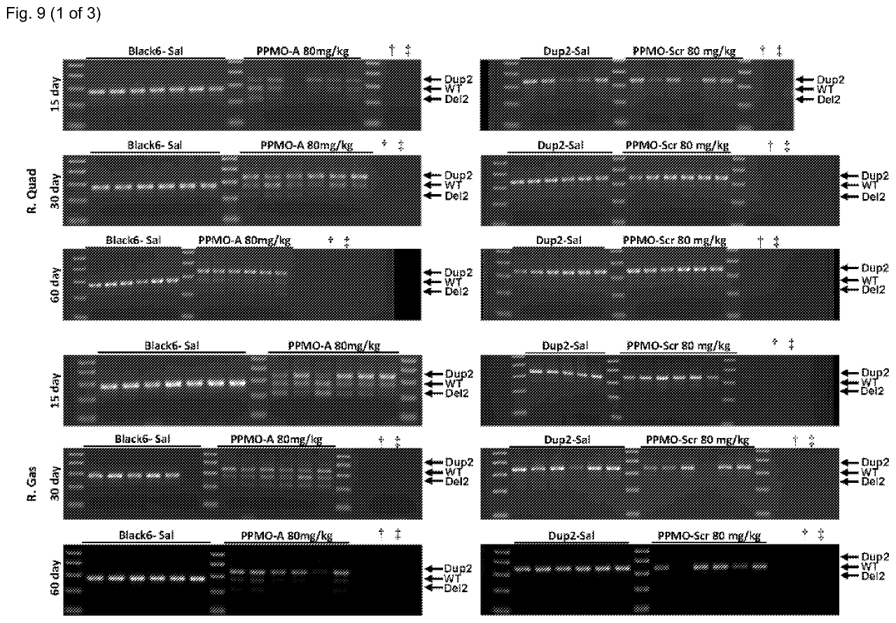

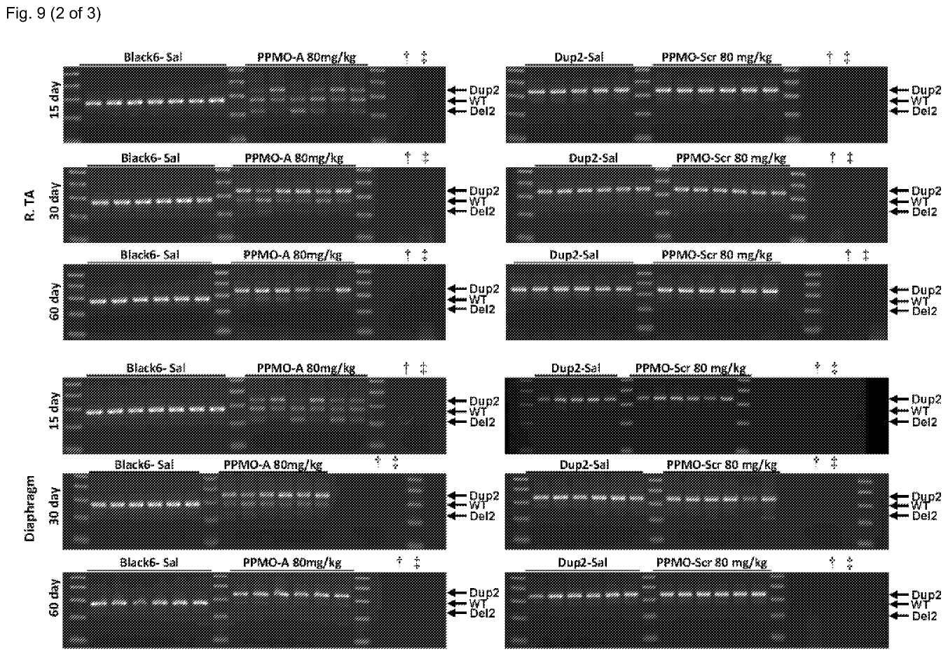

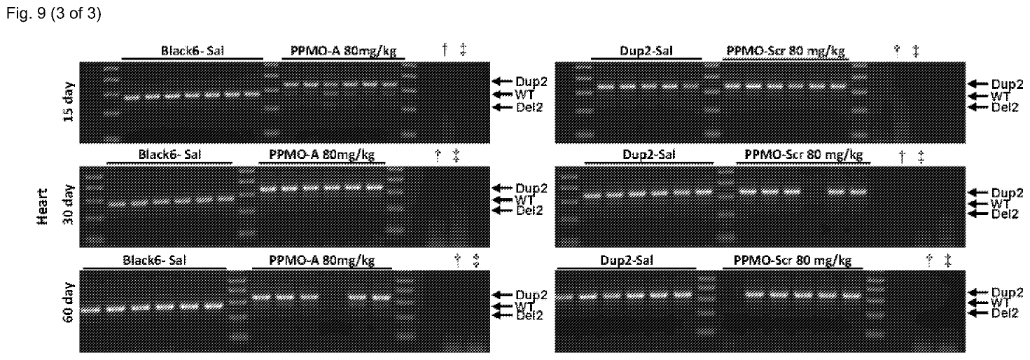

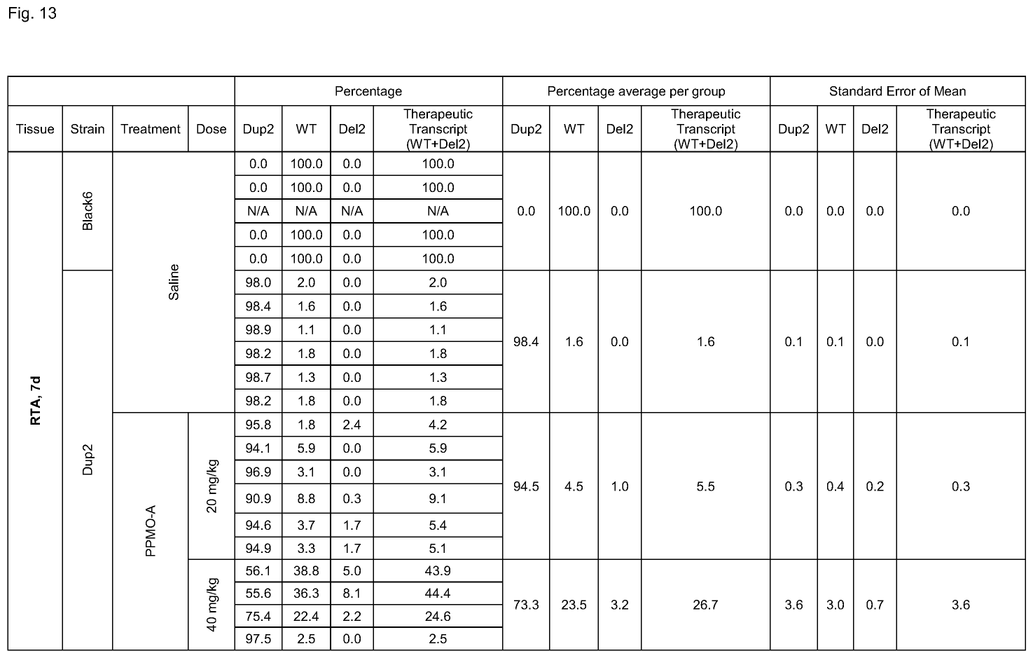

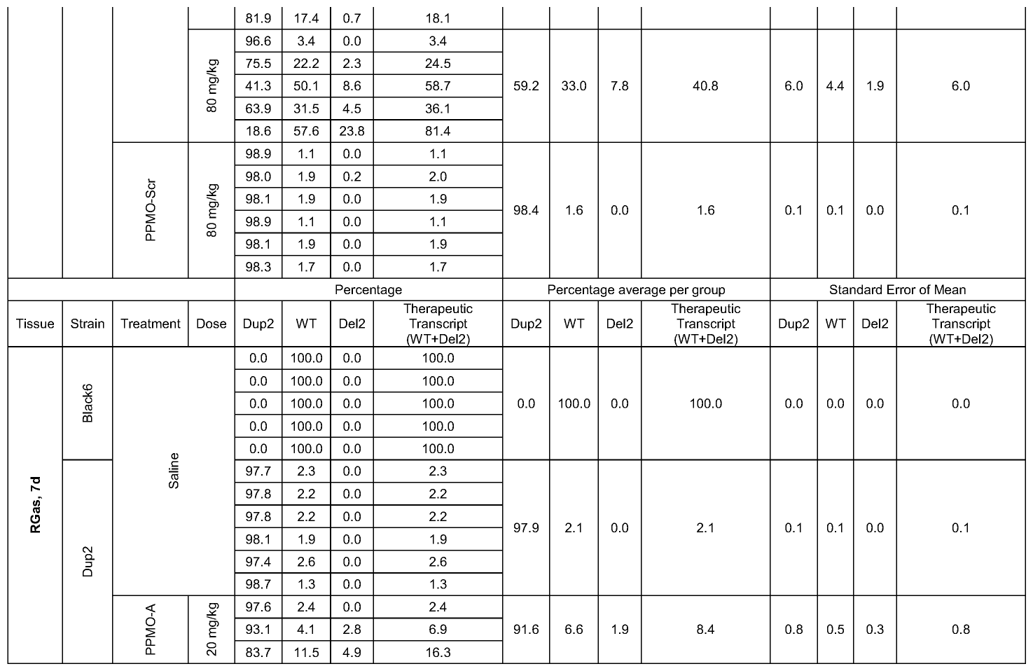

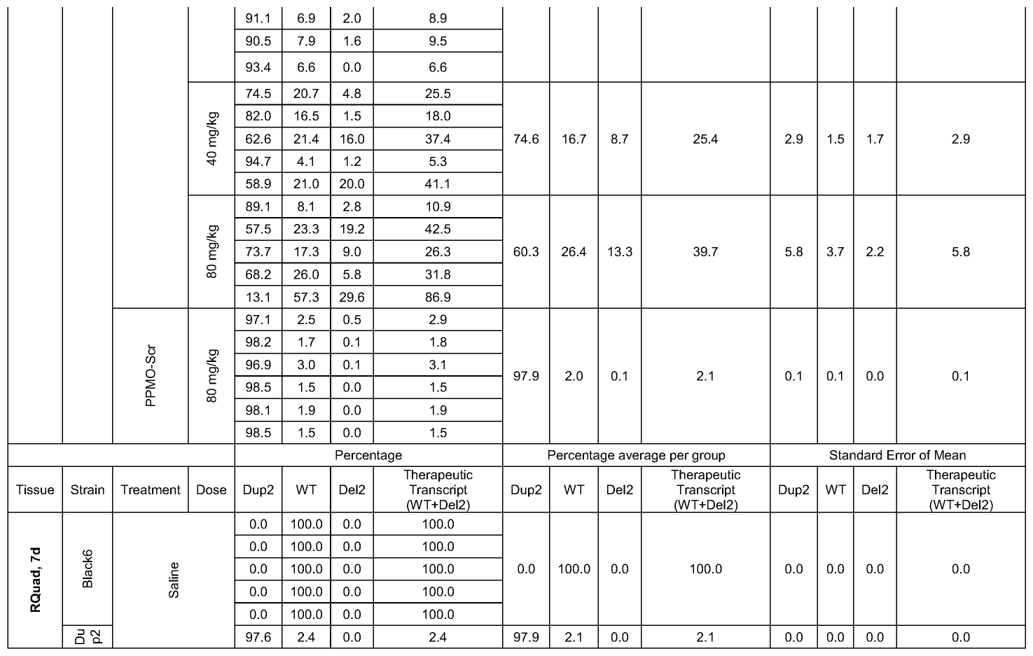

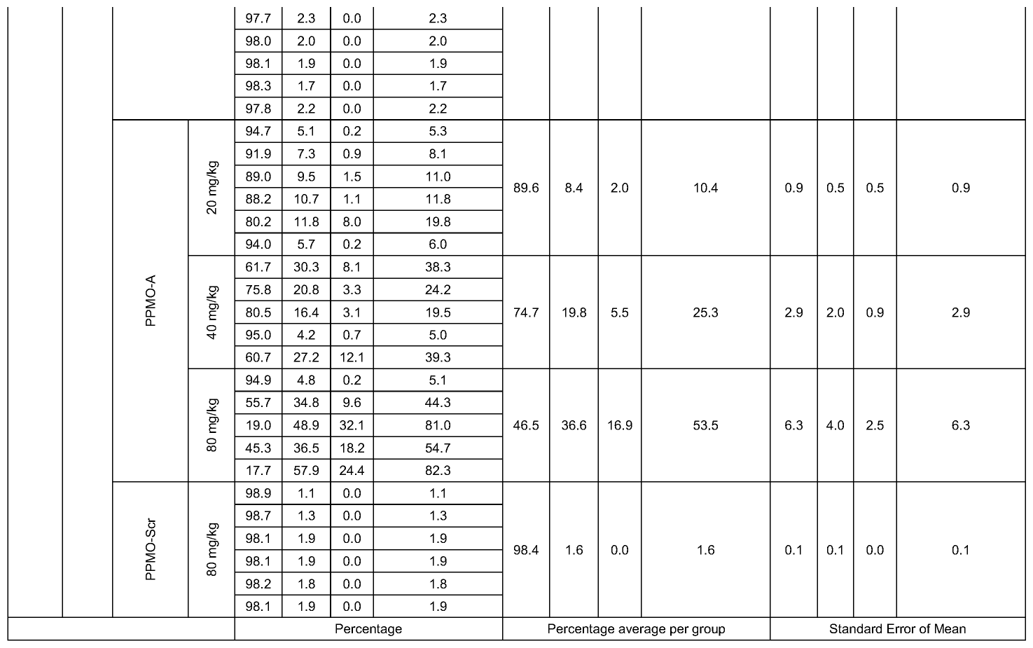

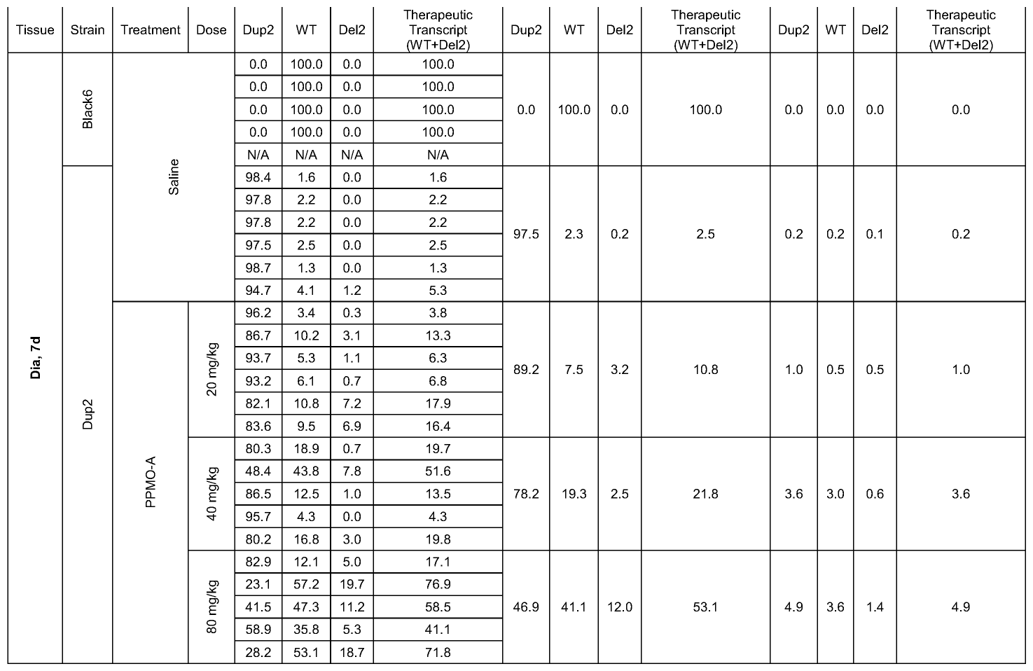

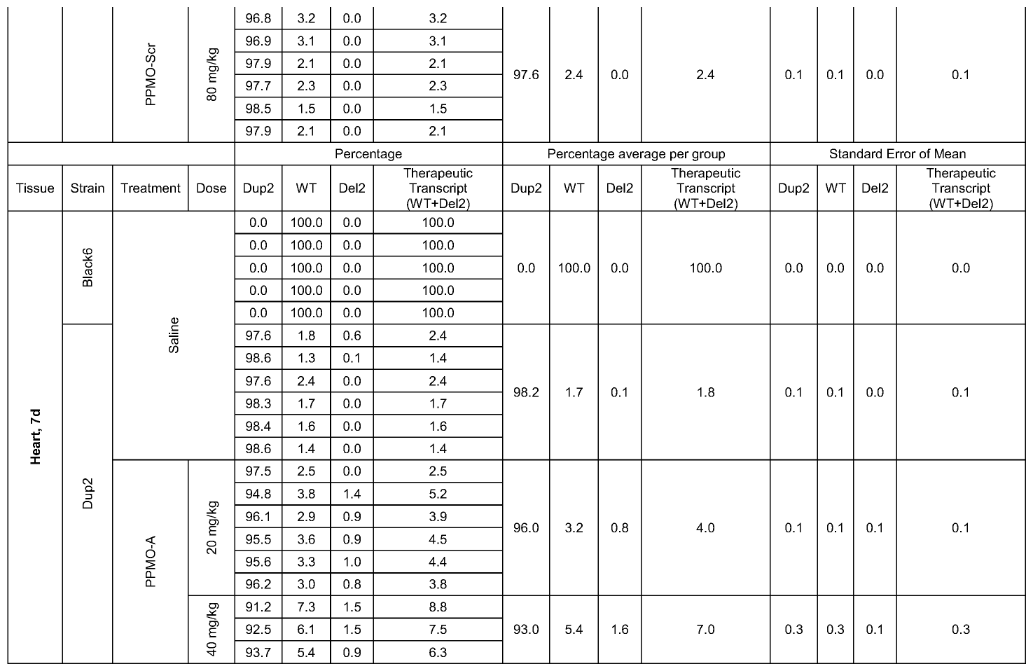

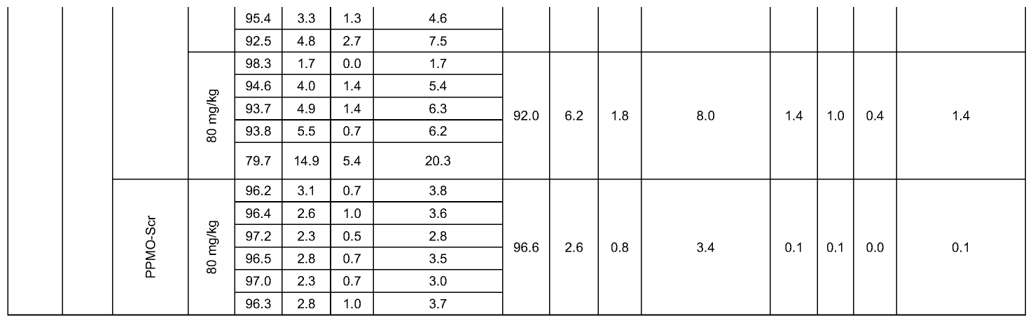

Seven days following the single injection, RT-PCR demonstrated the exon 2 exclusion resulted in accumulation of mRNA therapeutic transcripts, which include the wild-type (WT) and zero copies of exon 2 (Del2) in all tested skeletal muscles (FIGS. 1, 8, and 9). RT-PCR detected most robust DMD exon 2 exclusion at the mRNA level seen in quadriceps (Quad) at all doses (FIGS. 2, 13, and 14). In addition, tibialis anterior (TA), gastrocnemius (Gas) and diaphragm (Dia) had high ability to skip exon 2 in Dup2 treated mice, showing accumulation of total therapeutic transcripts (WT+Del2) at the highest PPMO-A1 dose in those tissues (FIG. 8 and FIG. 9).

DMD exon 2 quantification analysis revealed a significant increase of total therapeutic transcripts in a dose-dependent manner (FIGS. 2, 13, and 14), showing that PPMO-A1 at the minimal (20 m/kg) and middle (40 mg/kg) tested doses results in an average of 8.8% and 24.8% total therapeutic transcript, respectively, whereas animals treated at the dose of 80 mg/kg had higher levels of exon 2 skipping, representing an average 46.7% WT+Del2 therapeutic transcripts.

The minimal level of exon 2 exclusion was detected in the heart (FIGS. 2, 13, and 14) with no more than 8% total therapeutic transcript seen at the highest PPMO-A1 dose. The RT-PCR data also confirmed the similar level of endogenous skipping of exon 2 observed in Dup2 mice treated with either saline or PPMO-Scr, showing around 2% therapeutic transcript in all tested, including the heart, tissues, and suggesting that the PPMO-Scr is an appropriate negative control for this study since the antisense sequence in PPMO compound does not specifically target exon 2 and no specific effects related to the chemical modification was revealed.

### Example 3

### Single PPMO-A1 Treatment Rescued Dystrophin Expression in Skeletal Muscles but not in the Heart

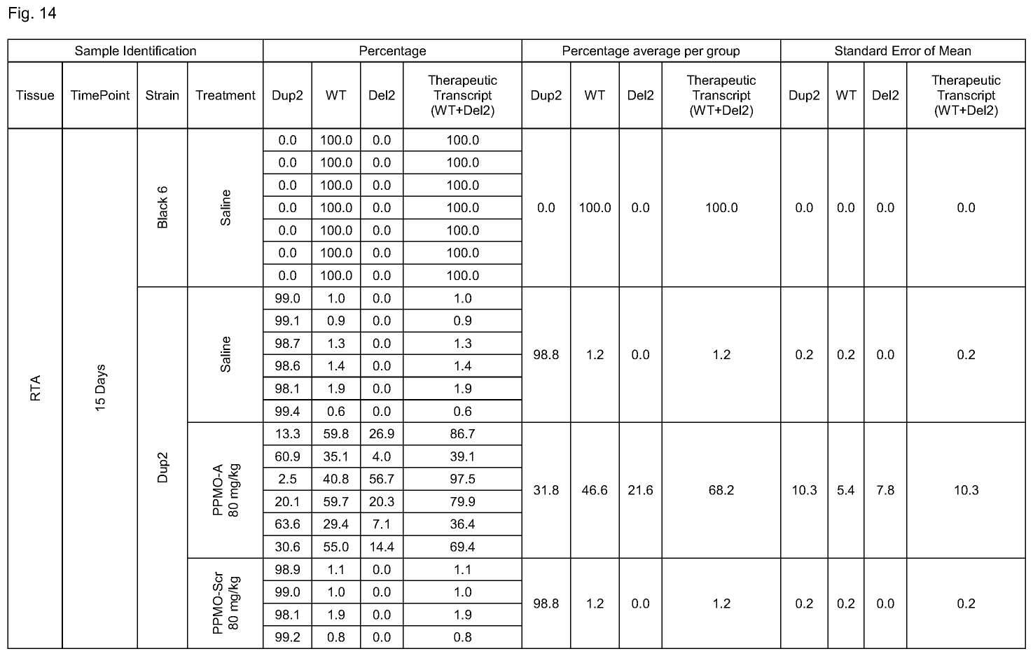

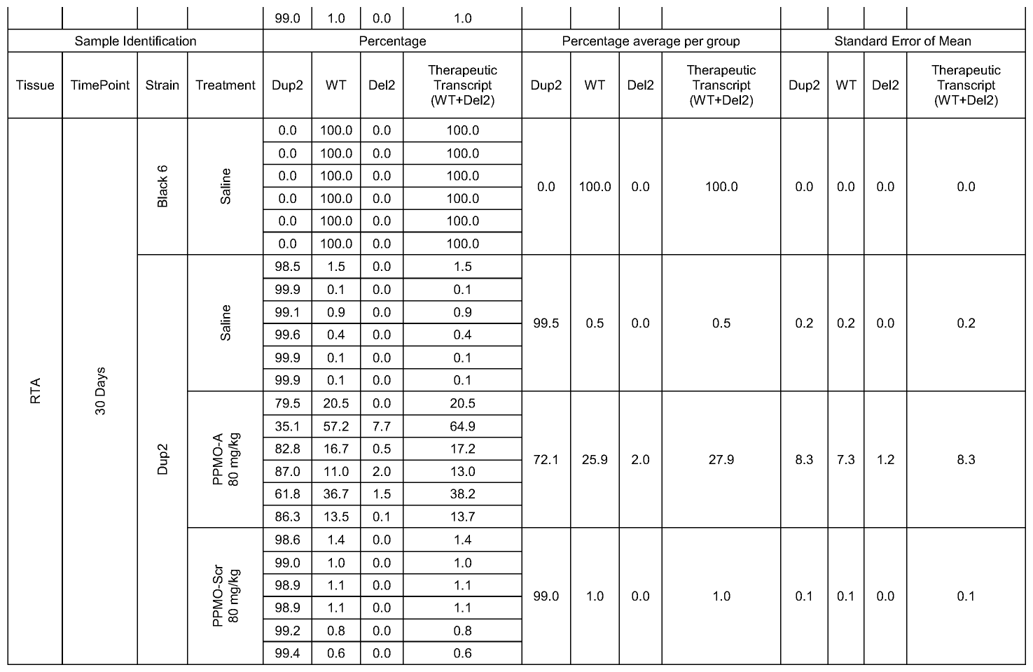

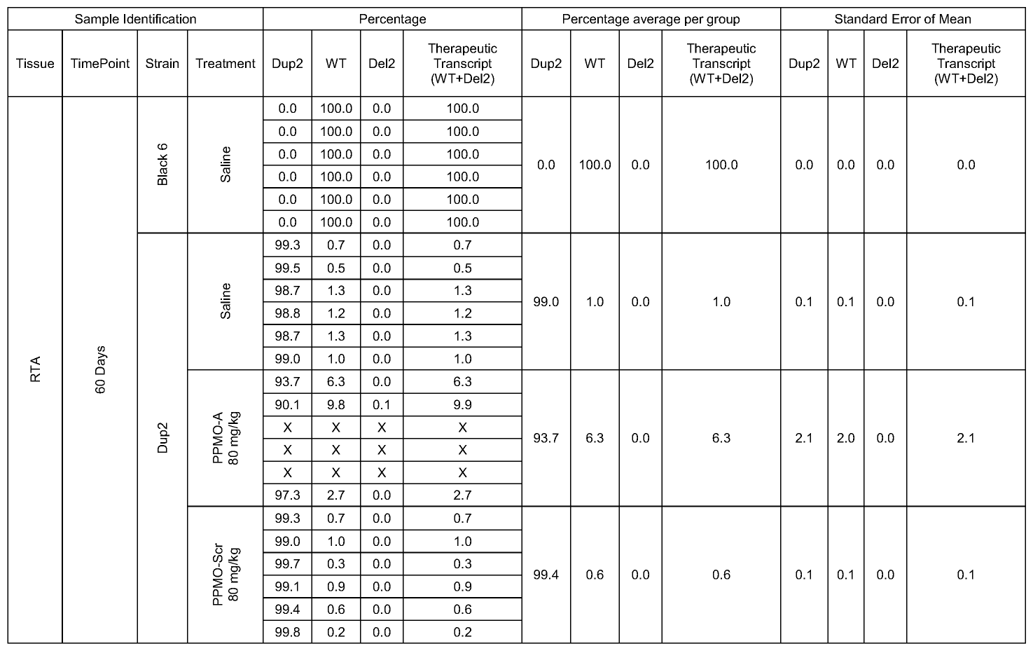

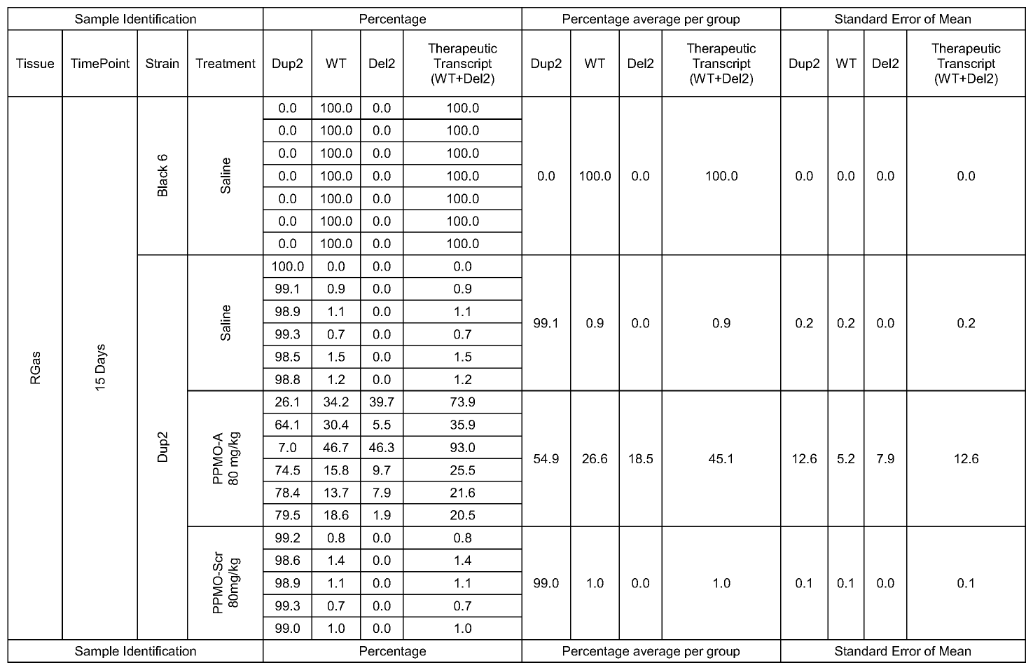

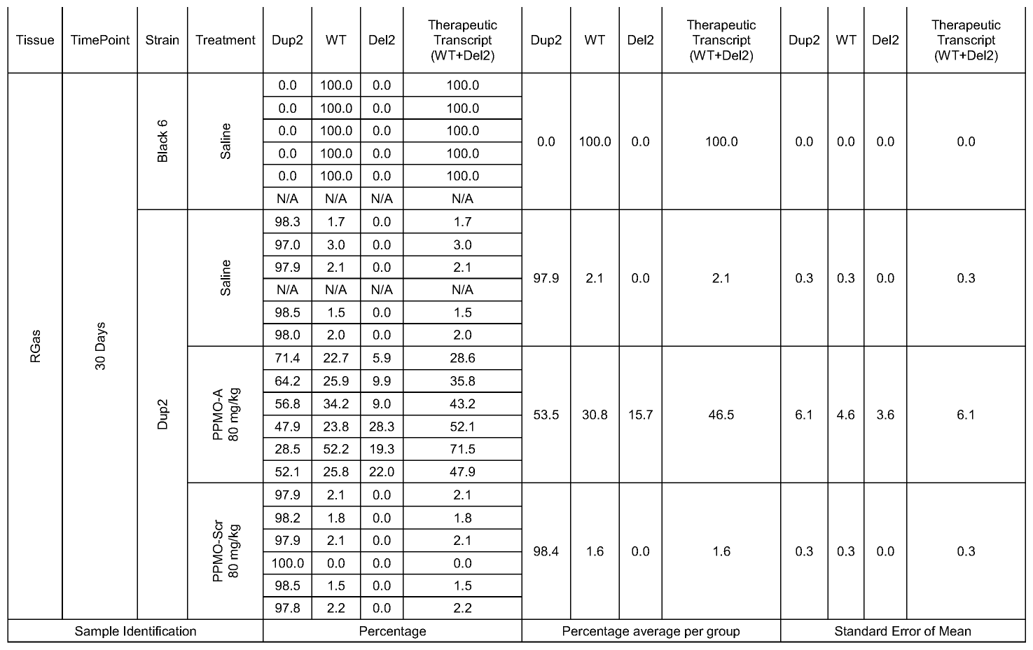

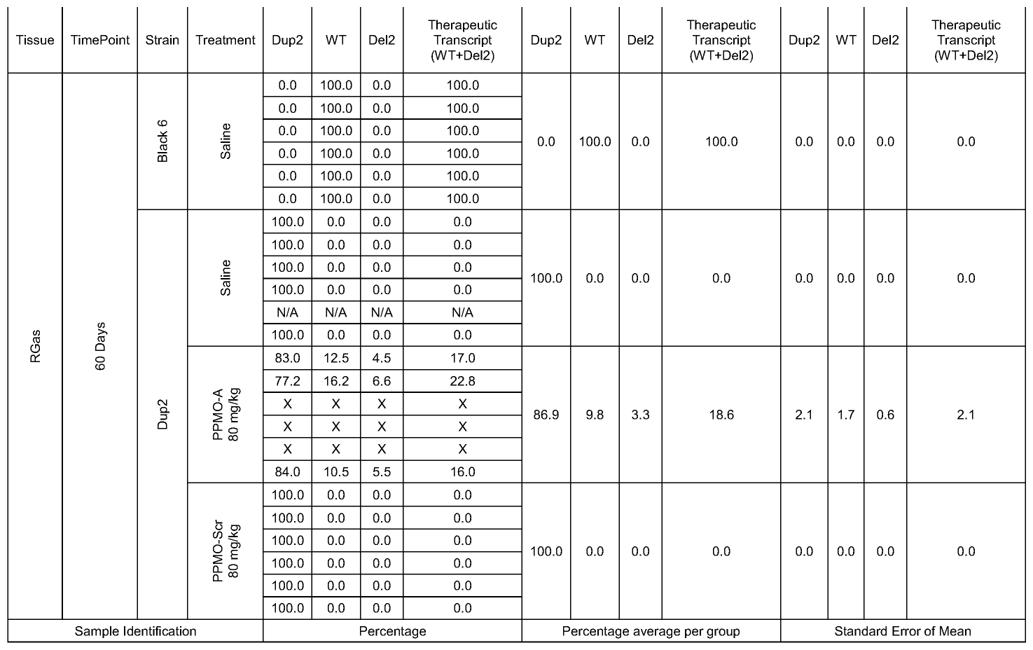

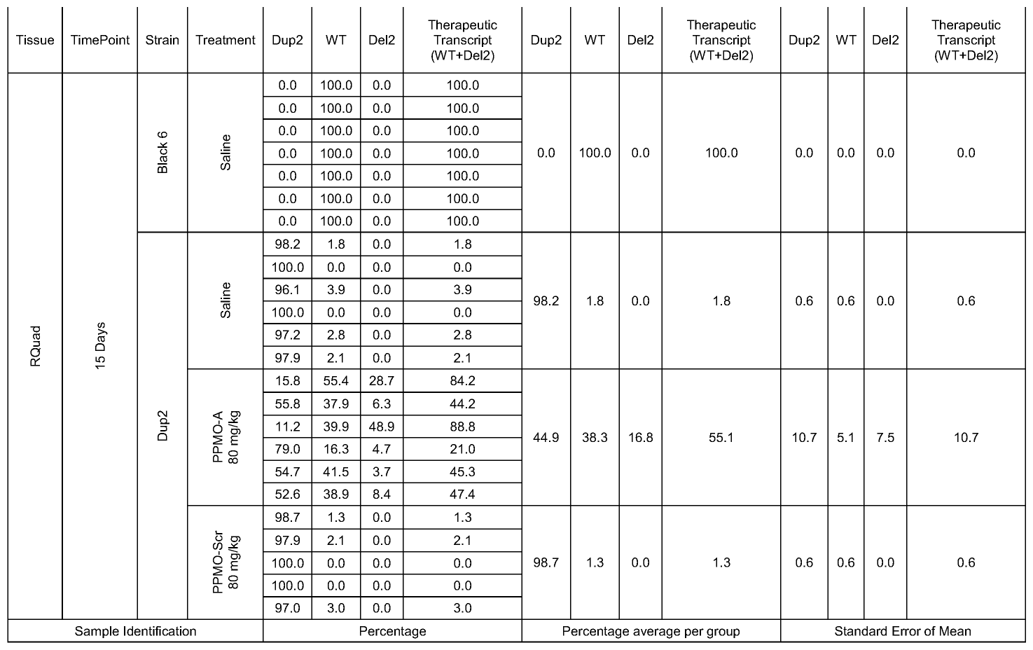

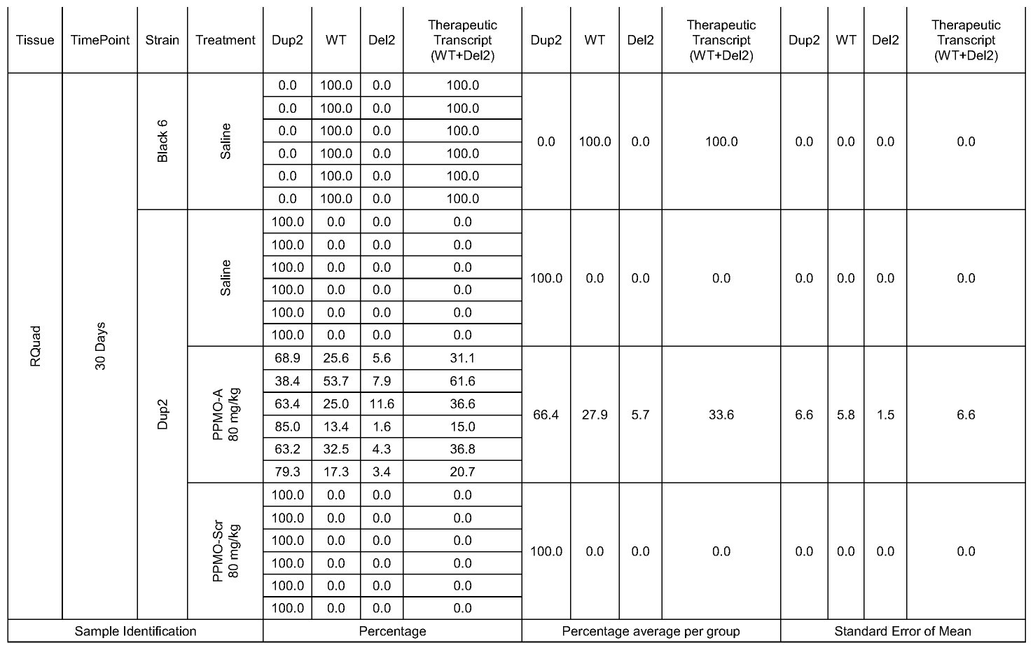

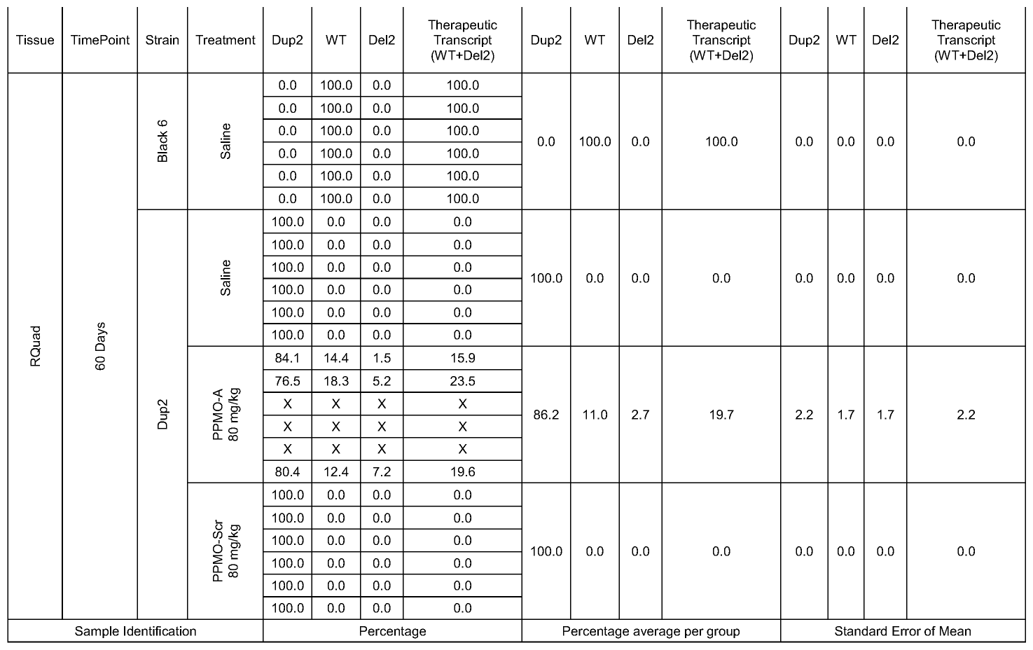

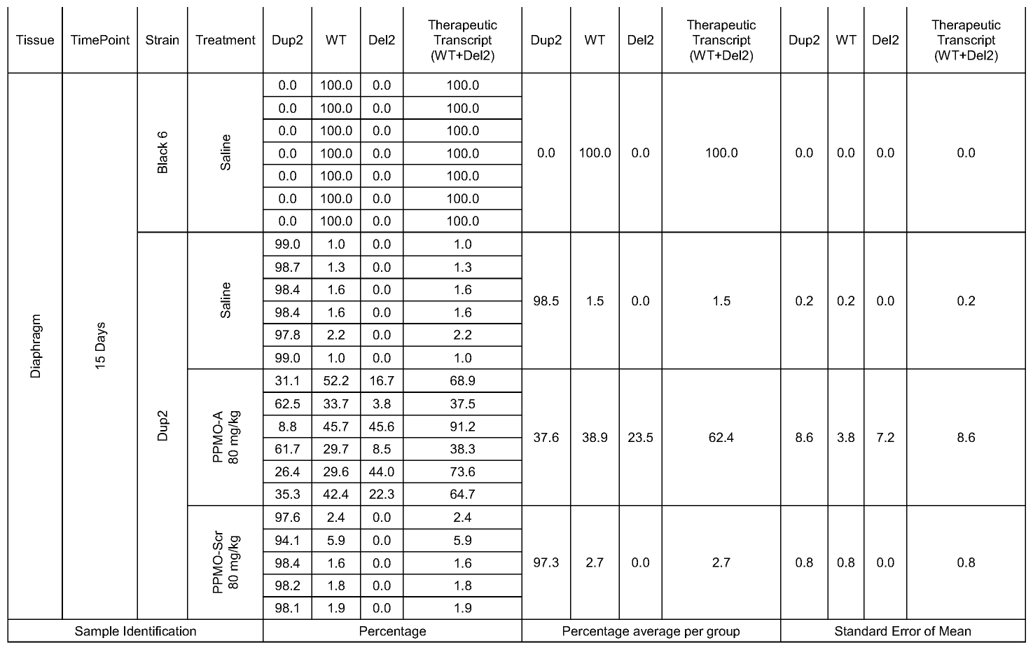

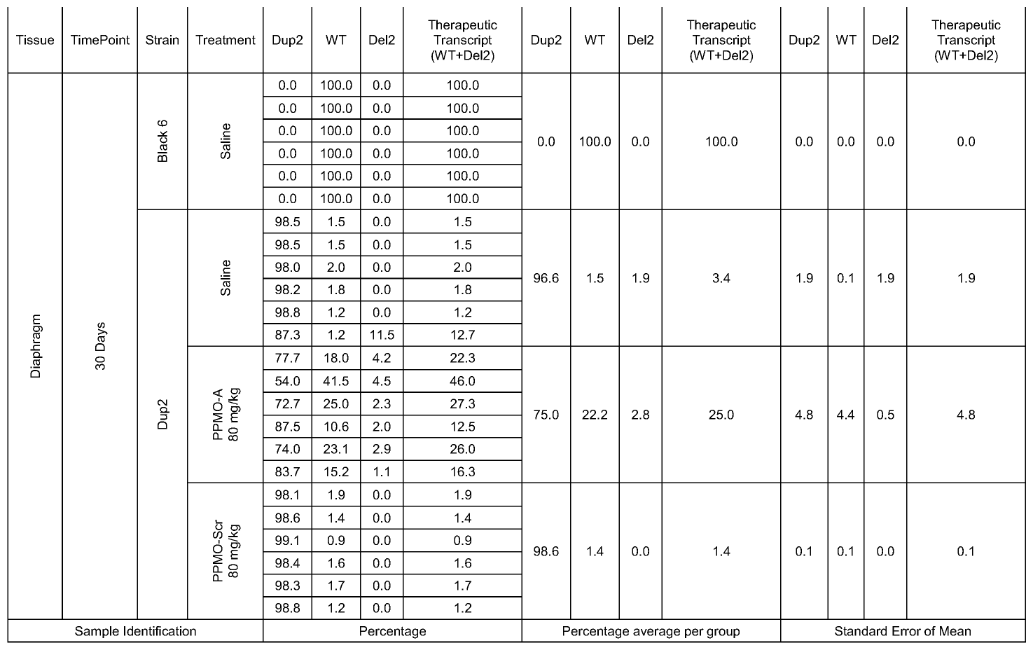

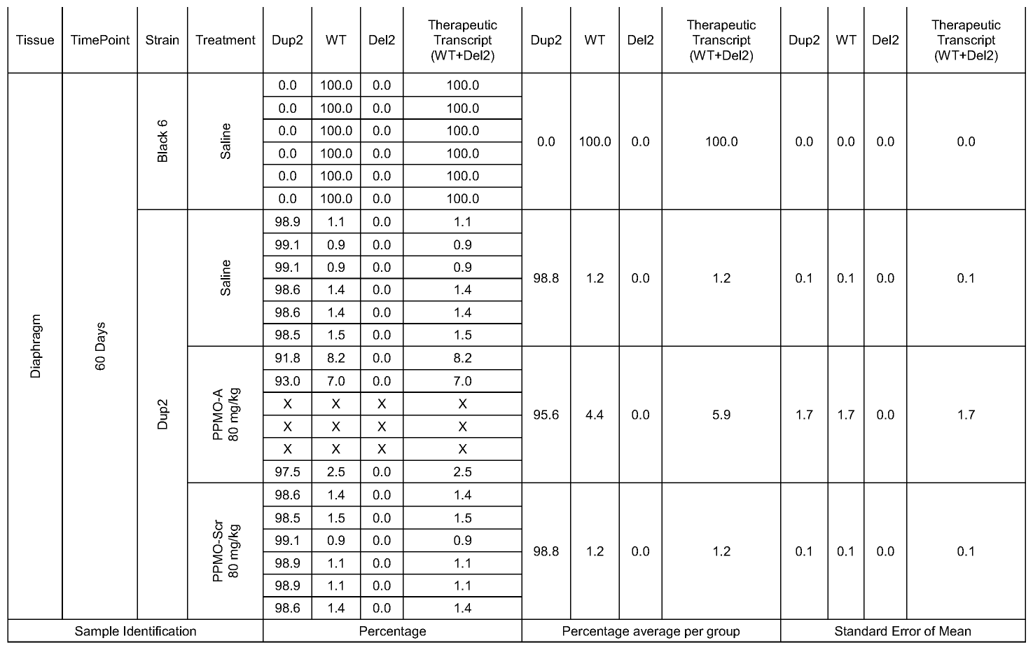

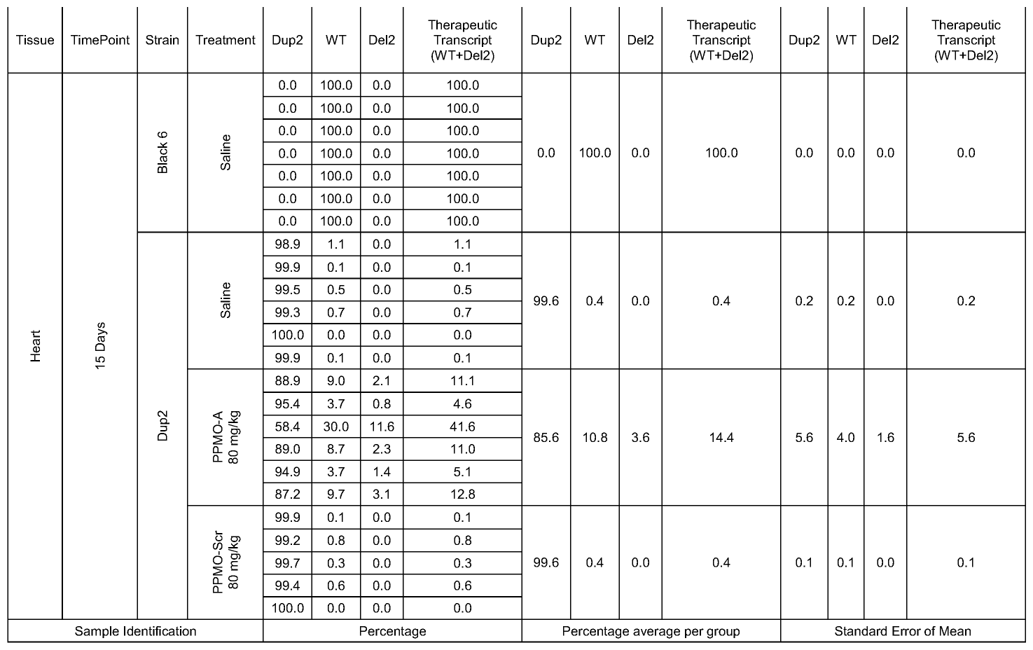

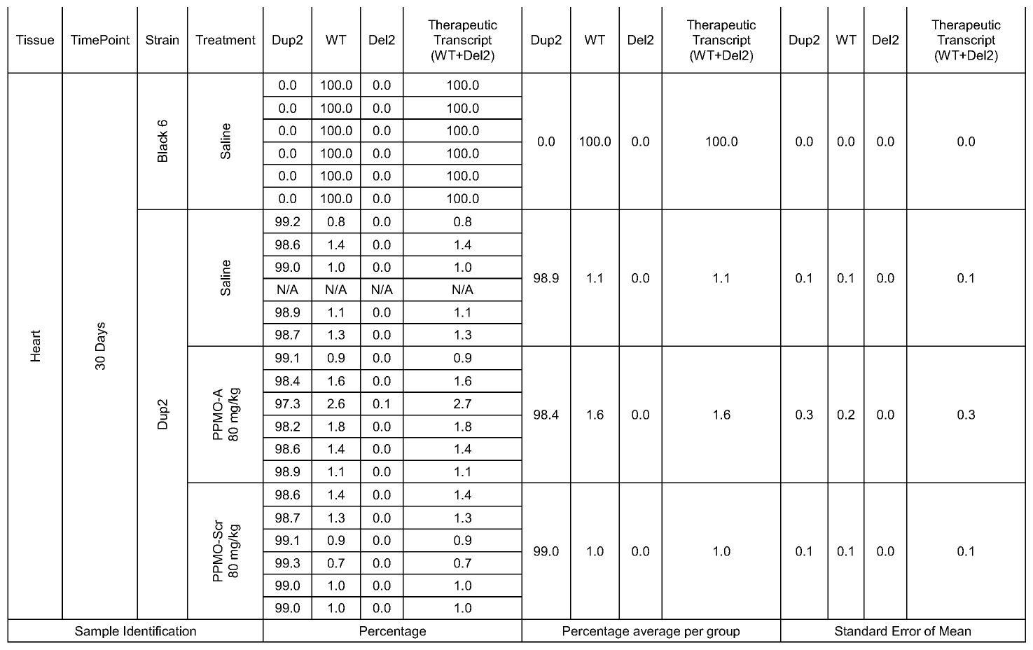

To assess whether the efficiency of PPMO-A1 compound to skip exon 2 was affected by time, a multiple point study was carried out in which exon 2 exclusion was evaluated in Dup2 mice treated with either PPMO-A1 or PPMO-Scr at the dose of 80 mg/kg or saline as a control and sacrificed 15, 30 and 60 days after treatment (FIGS. 1 and 14, and Table 4). C57BI/6 mice injected with saline were utilized as a control group being sacrificed at the same time-points as Dup2 groups.

TABLE 4
Time-Point Study Design.

| | | | | Doses | In-Life Duration |
|---|---|---|---|---|---|
| Strain | Sex | Compounds | n | (mg/kg) | (days) |
| Dup2 | Male | Saline | 6 | N/A | 15 |
| | | | 6 | | 30 |
| | | | 6 | | 60 |
| Dup2 | Male | PPMO-A1 | 6 | 80 | 15 |
| | | | 6 | | 30 |
| | | | 4 | | 60 |
| Dup2 | Male | PPMO-Scr | 5 | 80 | 15 |
| | | | 6 | | 30 |
| | | | 6 | | 60 |
| C57Bl/6 | Male | Saline | 7 | N/A | 15 |
| | | | 6 | | 30 |
| | | | 6 | | 60 |

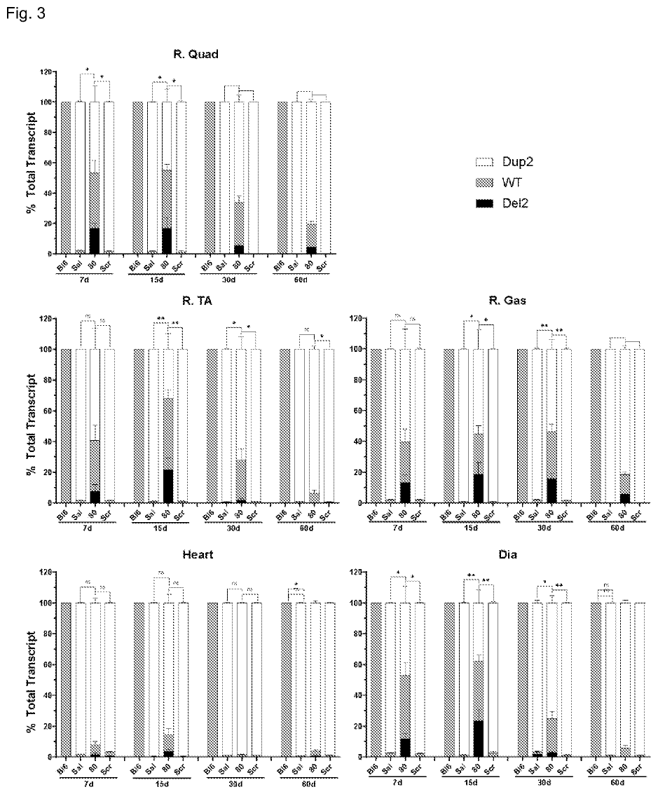

RT-PCR data are summarized in FIG. 3, including seven day results for PPMO-A1 at the high dose from the dose escalation study. The most robust exon 2 skipping was observed at 15 days post single PPMO-A1 administration in all tested skeletal muscles with a range from 45-55% (seen in the Gas and Quad muscles) to 62-68% (in the Dia and TA muscles). The PPMO-A1 uptake was still less pronounced in the heart, reaching up to 14.4% total therapeutic transcript 15 days after treatment. The degree of skipping was significantly reduced in time in all tested tissues, showing an average of 8.5% total therapeutic transcript 60 days post PPMO-A1 injection.

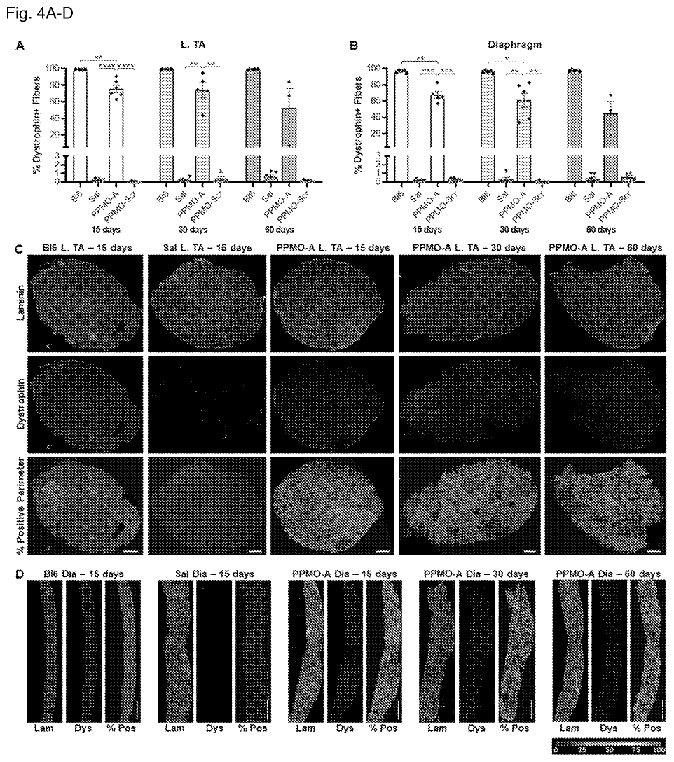

The quantification of immunofluorescence (IF) staining (FIG. 4A-B) revealed a high level of dystrophin-positive fibers observed in both TA (74.3-75.7%) and Dia (61-67.7%) muscles at 15 and 30 days in PPMO-A1-treated groups. The percentage of dystrophin-positive fibers showed a declining trend at 60 days post-treatment, but still quantified as 52.9% in TA and 45% in Dia tissues, respectively. In general, ≤1% positive fibers were detected in saline and PPMO-Scr Dup2 treated groups, whereas C57BI/6 mice confirmed 96.7-99% dystrophin-positive fibers in both TA and Dia tissues.

The representative whole-section images of both TA muscle (FIG. 4C) and Dia (FIG. 4D) showed exclusively localized dystrophin protein (red channel) at the sarcolemmal membrane when co-stained with laminin (green channel). The color-coded heatmaps were generated to display the percent of the perimeter with dystrophin-positive pixels for each muscle fiber, where fibers showing ≥70% dystrophin-positive perimeter were considered overall as dystrophin-positive.

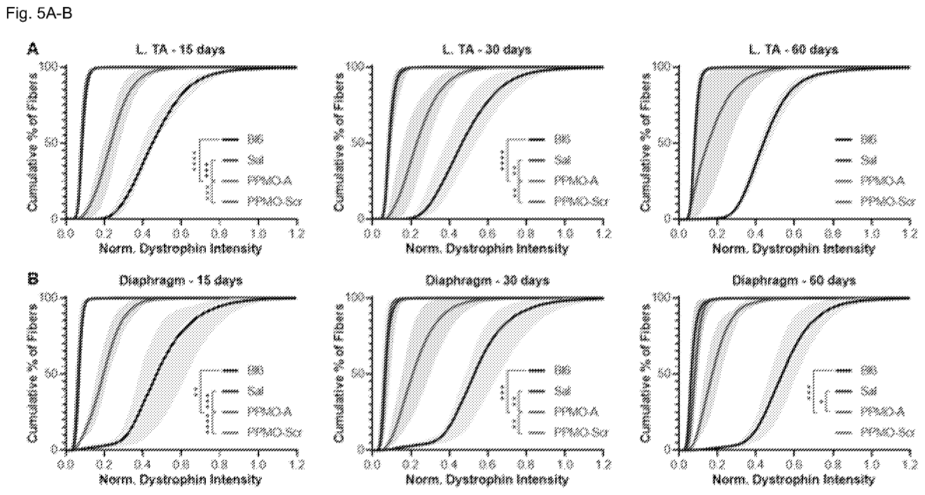

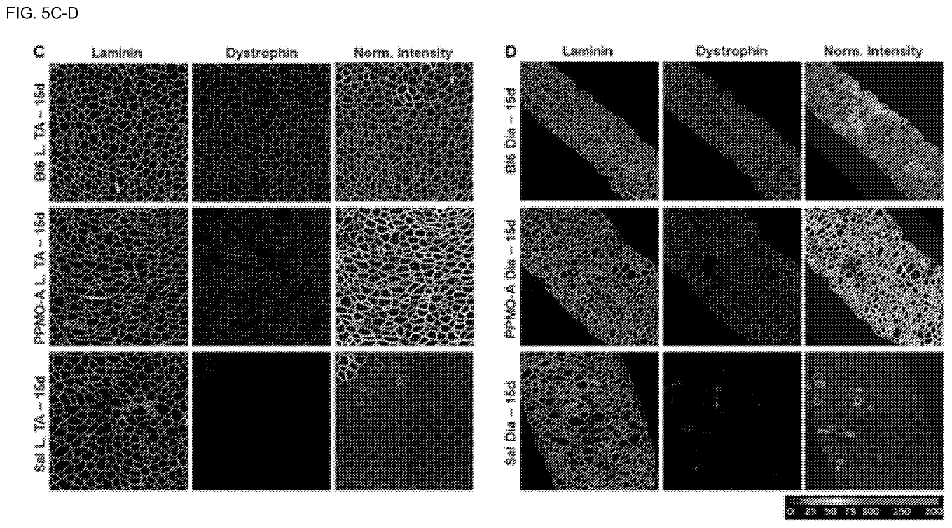

Dystrophin signal intensity quantified for each fiber in TA and diaphragm sections (FIG. 5A-D) further confirmed effective dystrophin restoration by PPMO-A1 treatment. In TA sections of Dup2 mice treated with PPMO-A1, the mean fiber dystrophin intensity reached approximately 50-51% of BI6 mean dystrophin intensity at 15- and 30-days post-treatment, and 38% at 60 days (FIGS. 5A and 5C). Dia sections showed a similar trend in fiber dystrophin intensity after PPMO-A1 treatment, with approximately 41-42% dystrophin intensity at 15 and 30 days after PPMO-A1 treatment and 34% dystrophin intensity at 60 days after treatment (FIGS. 5B and 5D). Mean dystrophin channel signal intensity in fibers from saline and PPMO-Scr treatment groups remained≤19% in TA sections and ≤16% in Dia, reflecting a combination of tissue autofluorescence, very low baseline dystrophin expression, and a small number of revertant fibers. Additional color-coded heatmaps reflecting fiber dystrophin intensity are shown for representative regions from TA and diaphragm sections.

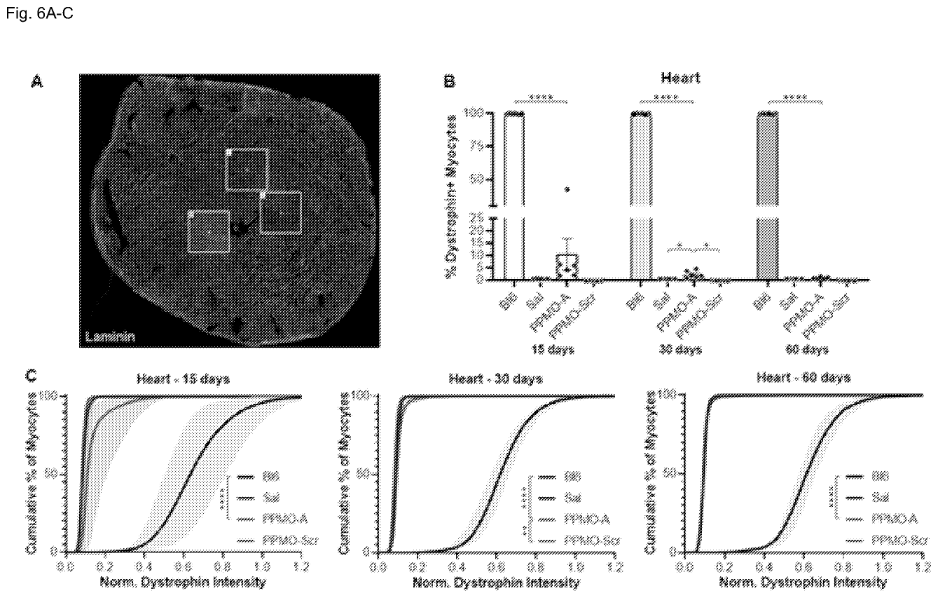

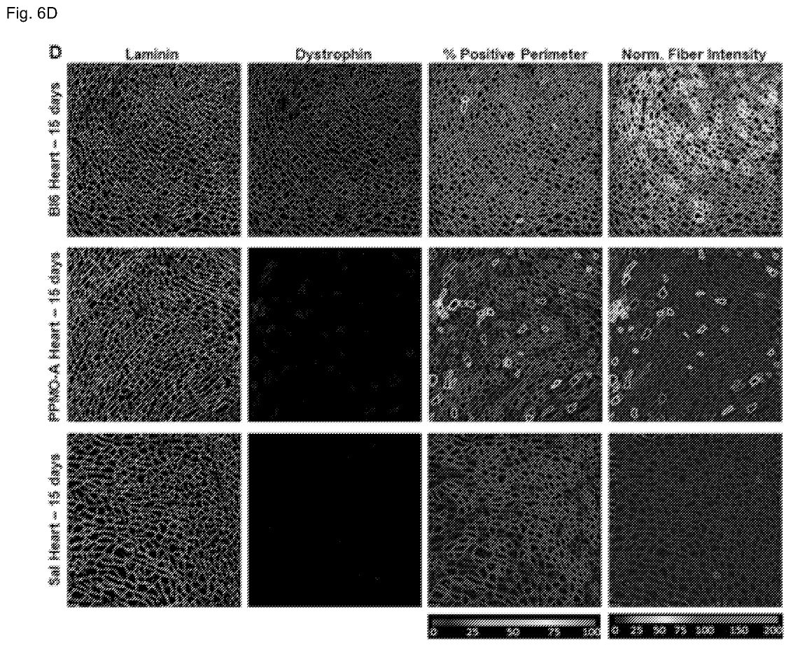

PPMO-A1 efficacy was much lower in the heart. Such efficacy in the heart was analyzed by quantifying three square regions of interest (ROIs) of cross-sectional myocytes per heart to ensure successful sarcolemmal detection (FIGS. 6A and 6D). Aside from a single outlier displaying 42.3% dystrophin-positive myocytes (was not excluded from the graph), PPMO-A1 treated hearts showed, on average, 4.0% dystrophin-positive myocytes at 15 days and 2.3% at 30 days after treatment. However, this level of expression was still significantly higher than in saline and PPMO-Scr treatment groups at any time points, which showed no more than 0.1% dystrophin-positive myocytes (FIGS. 6B and 6D). Cardiac myocyte dystrophin intensity showed a similarly modest response to PPMO-A1 treatment, with 16-20% dystrophin intensity in the treated Dup2 compared to C57BI/6 hearts at 15 and 30 days, and 13-15% in Dup2 that received saline or PPMO-Scr. By 60 days post-treatment, no trend toward increased dystrophin expression in the heart was observed in any Dup2 treatment groups (FIG. 6C).

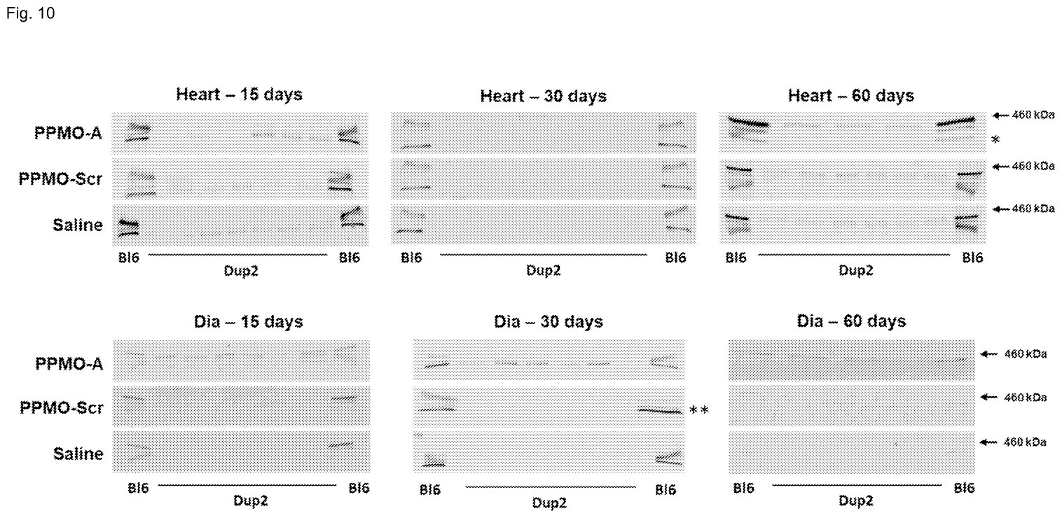

Western blots images of left TA muscle (FIG. 7A), Dia and heart tissues (FIG. 10) and their quantitative analysis (FIG. 7B) revealed results similar with the IF pattern showing that a single PPMO-A1 injection rescued significant dystrophin synthesis, resulting in 20-27% dystrophin protein detected at 15 and 30 days in both TA and Dia tissues, and 6.6-27% in heart between 15-30 days. At 60 days, TA, Dia and heart demonstrated 22.7%, 14.8% and 5% restored dystrophin, respectively. Furthermore, for all control groups, i.e., Dup2 treated with PPMO-Scr and saline, dystrophin expression did exceed≤5% at all timepoints (FIG. 7B).

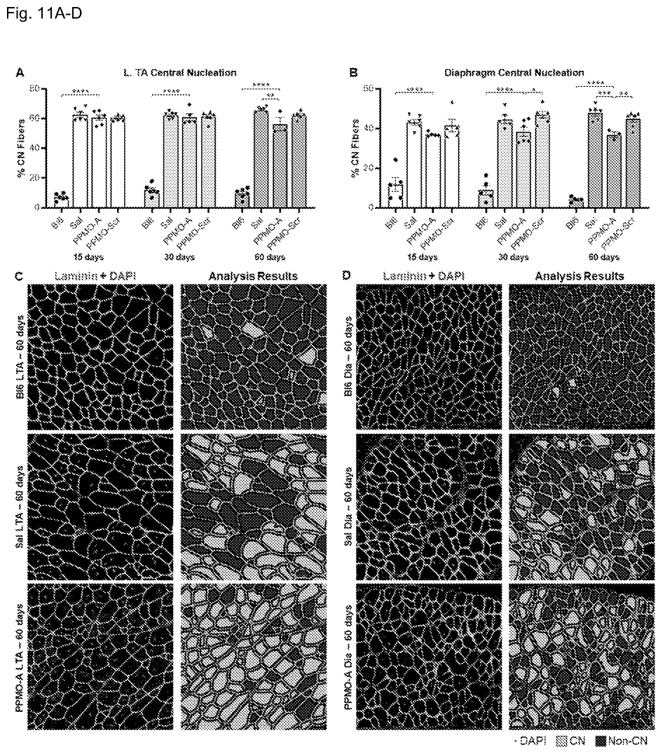

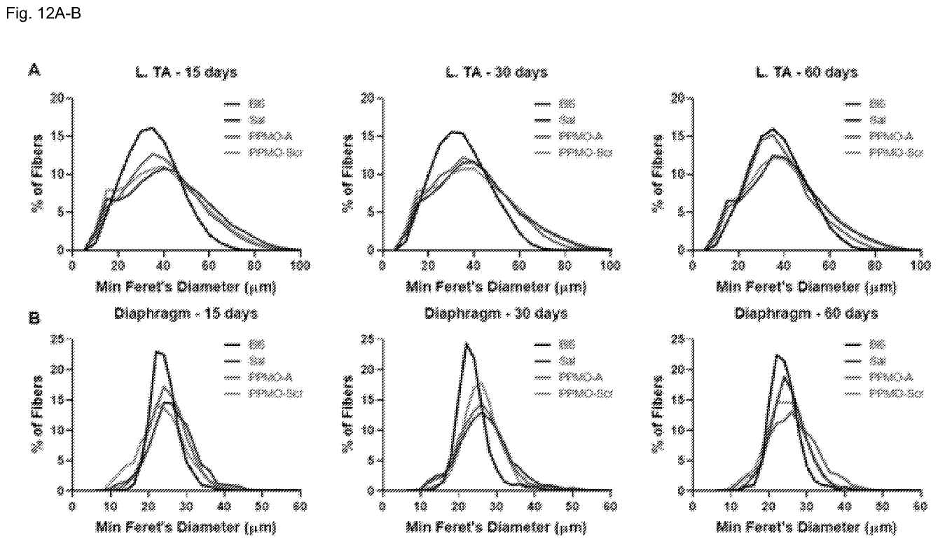

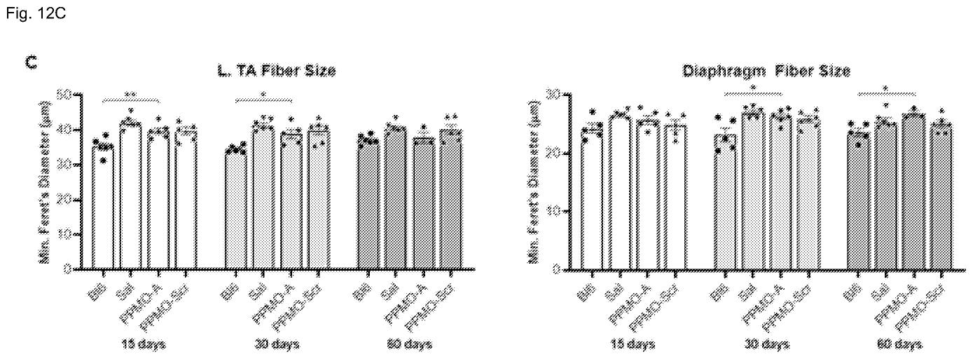

Central nucleation (FIG. 11A-D) showed a significant response to PPMO-A1 treatment in Dup2 mice by 60 days post-injection in both TA and diaphragm sections. Mean central nucleation in Dup2 TAs 60 days after receiving Saline or PPMO-Scr treatment was 65.9% and 62.6% respectively, while the PPMO-A1 treatment group showed 56.2% centrally nucleated fibers. Similarly, diaphragms from Dup2 mice that received Saline or PPMO-Scr showed 45-48% centrally nucleated fibers at 60 days, while PPMO-A1-treated diaphragms showed 36.9% central nucleation. Interestingly, central nucleation in the diaphragm showed an earlier and more pronounced response to PPMO-A1 treatment than in TA sections. Fiber size was also assessed in TA and diaphragm sections, shown as the minimum Feret's diameter (FIG. 12A-C), but no significant treatment effect on fiber size could be detected at the tested time points.

This study shows that the PPMO-A1 31-mer sequence, a novel cell-penetrated PMO conjugate targeting the splice acceptor site of exon 2 DMD gene, is a promising therapeutic agent in the treatment of DMD patients with Dup2 mutations. The tested PPMO approach shows that PPMO-A1 can be to induce robust exon 2 skipping in skeletal muscles, including the diaphragm, and leads to restoration of full-length dystrophin as well as its truncated, but functionally active isoform in a Dup2 mouse model. This approach can become even more feasible in further treatment of patients previously treated with an AAV approach which may have had an insufficient level of dystrophin expression, and/or of patients with pre-existing anti-capsid immunity, which makes them unable to be enrolled in AAV-mediated therapies.

Results presented herein suggest that the delivery of PPMO-A1 compound including, but not limited to, systemic delivery, has a great promise as a therapeutic agent resulting in a significant rescue of dystrophin in skeletal muscles.

### Example 4

Expression of Dystrophin after Treatment of Human Patients with Exon 2 Duplications

Following dose finding studies in mice and after demonstrating lack of toxicity in non-human primates, a first-in-human clinical is initiated in patients having exon 2 duplications of their DMD gene. As discussed herein above, if the targeted exon is skipped, a truncated, yet functionally active dystrophin is expressed protein is expressed by this therapy.

Patients having exon 2 duplications (the Dup2 mutation) are administered the PPMO-A1 31-mer sequence, as described herein above, at one or more intravenous doses in the range of about 1 mg/kg to about 100 mg/kg. The PPMO-A1 sequence targets the splice acceptor site of exon 2 of the DMD gene. Because these patients have exon 2 duplications, the PPMO-A1 construct will bind to the exon 2 splice acceptor site on the DMD gene and exclude exon 2 from the dmd transcript, resulting in a truncated yet functional form of dystrophin in muscle cells of the patient.

### Example 5

### Single PPMO-A1 Treatment Rescued Dystrophin Expression in Skeletal Muscles but not in the Heart

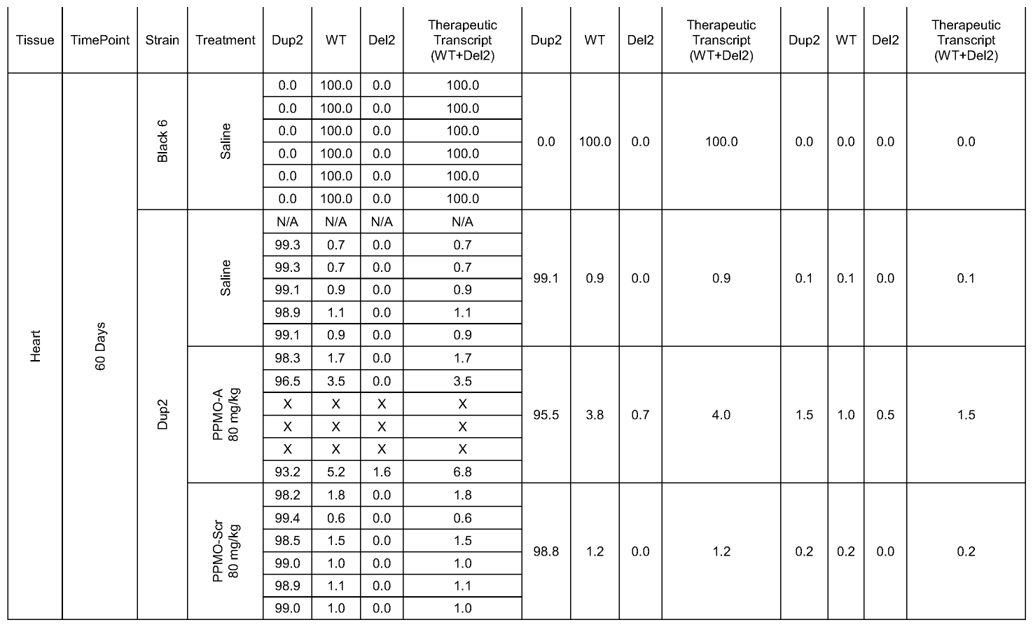

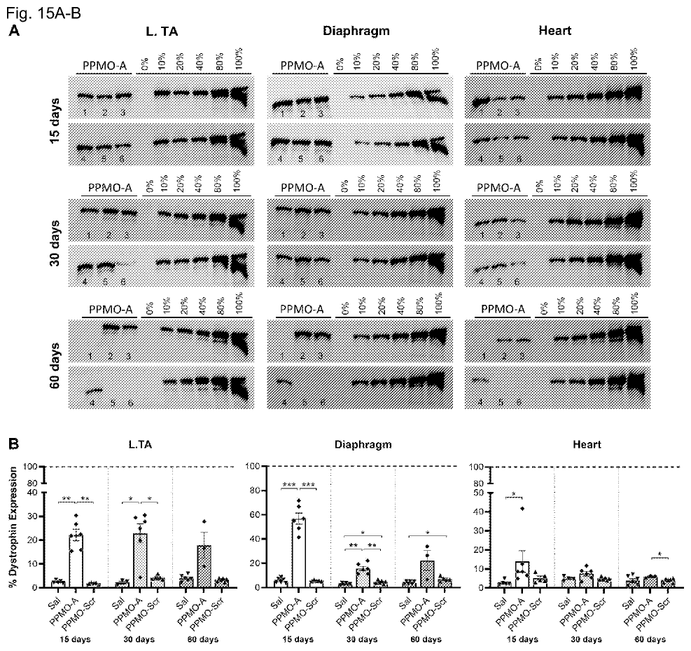

To assess whether the efficiency of PPMO-A1 compound to skip exon 2 was affected by time, a multiple point study was carried out in which exon 2 exclusion was evaluated in Dup2 mice treated with either saline (control), PPMO-A1, or PPMO-Scr (control) at the dose of 80 mg/kg and sacrificed 15, 30 and 60 days after treatment (FIG. 15A-B and Table 4). C57BI/6 mice injected with saline were utilized as a control group being sacrificed at the same time-points as Dup2 groups.

FIG. 15A-B shows the results of dystrophin protein expression analysis after treatment of Dup2 mice with PPMO-A1. FIG. 15A shows the Western blot expression of dystrophin from muscle lysates in left tibialis anterior (L.TA), diaphragm, and heart at 15, 30, and 60 days after treatment with PPMO-A1. Western blots for dystrophin were performed on muscle lysates that were loaded to match total protein content as determined by a DC assay. Each blot included 3 test samples and a 6-point standard curve made by diluting pooled wild type tissue samples dystrophin from six C57BI/6 mice in dystrophin-null lysate from age-matched Dup2Del18-41 mice, a spontaneous mouse model that developed from the Dup2 mouse model and a spontaneous deletion of exons 18-41 of the dmd gene. Dup2Del18-41 mice do not express any dystrophin compared to the Dup2 mice that express ˜5 of dystrophin. FIG. 15B shows quantitative analysis of percent dystrophin expression in the muscle lysates in left tibialis anterior (L.TA), diaphragm, and heart at 15, 30, and 60 days after treatment with saline (control), PPMO-A1, or PPMO-Scr (control). Dystrophin bands were analyzed using Image Lab software by quantifying the area under the curve of individual mouse samples against a linear regression line fit to the standard curve bands on each blot (R²≥0.927 for each standard curve). Samples 1, 5 and 6 from the 60-day group were found to have a spontaneous deletion of Dmd exons 18-41 (Dup2Del18-41), and were thus excluded from quantification. All blot images representing PPMO-A1-treated samples are shown. Statistical results reflect Welch's ANOVA with Dunnett's T3 multiple comparison test; *p<0.05; **p<0.01; ***p<0.001.

Western blot quantification showed that a single PPMO-A injection rescued significant dystrophin production in skeletal muscle, but showed reduced efficacy in the heart (FIG. 15A). PPMO-A1 treatment resulted in 22-23% mean dystrophin expression by Western blot in the TA 15-30 days after treatment, and 18% at 60 days (FIG. 15B). The diaphragm showed more robust peak expression of 57% at the 15-day time point, which declined to 16-22% at the 30- and 60-day points (FIG. 15B). The heart displayed a modest treatment effect of 14% mean dystrophin expression at 15 days, 7.6% at 30 days, and 5.8% at 60 days (FIG. 15B). Mean dystrophin expression by Western blot in Dup2 control groups treated with PPMO-Scr and saline remained below 7.0% in muscle and 5.2% in the heart at all timepoints (FIG. 15B).

This study shows that the PPMO-A1 31-mer sequence, a novel cell-penetrated PMO conjugate targeting the splice acceptor site of exon 2 DMD gene, can be used to induce robust exon 2 skipping in skeletal muscles, including the diaphragm. Results presented herein suggest that the delivery of the PPMO-A1 compound including, but not limited to, systemic delivery, has a great promise as a therapeutic agent resulting in a significant rescue of dystrophin in skeletal muscles.

While the disclosure has been described in terms of specific aspects, it is understood that variations and modifications will occur to those skilled in the art. Accordingly, only such limitations as appear in the claims should be placed on the disclosure.

All documents referred to in this application are hereby incorporated by reference in their entirety.

We claim:

1. An oligonucleotide comprising a nucleotide sequence selected from the group consisting of:
(a) a nucleotide sequence comprising at least 97%, at least 98%, at least 99% identity to the sequence set forth in any one of SEQ ID NOs: 1 and 2; and
(b) the nucleotide sequence comprising the sequence set forth in any one of SEQ ID NOs: 1 and 2.

2. An antisense oligonucleotide construct comprising a peptide conjugated to an oligonucleotide comprising a nucleotide sequence selected from the group consisting of:
(a) a nucleotide sequence comprising at least 80% identity to the sequence set forth in any one of SEQ ID NOs: 1 and 2;
(b) a nucleotide sequence complementary to the nucleotide sequence comprising at least 80% identity to the sequence set forth in any one of SEQ ID NOs: 1 and 2;
(c) a nucleotide sequence comprising the sequence set forth in any one of SEQ ID NOs: 1 and 2;
(d) a nucleotide sequence complementary to the nucleotide sequence comprising the sequence set forth in any one of SEQ ID NOs: 1 and 2; and
(e) a nucleotide sequence which binds to the sequence set forth in SEQ ID NO: 4.

3. The antisense oligonucleotide construct of claim 2, wherein the construct is a phosphorodiamidate morpholino oligomer (PMO) or a peptide-conjugated PMO (PPMO).

4. A composition comprising the
(a) the oligonucleotide of claim 1; or
(b) the antisense oligonucleotide construct of claim 2 or 3; and
a carrier, diluent, excipient, and/or adjuvant.

5. A method of treating, preventing or ameliorating a muscular dystrophy in a subject in need thereof comprising the step of administering to the subject an effective amount of
(a) the oligonucleotide of claim 1;
(b) the antisense oligonucleotide construct of any one of claims 2 and 3; or
(c) the composition of claim 4.

6. The method of claim 5, wherein the muscular dystrophy results from a 5′ mutation in the DMD gene.

7. The method of claim 6, wherein the 5′ mutation is a mutation involving any one or more of exons 1-5.

8. The method of claim 6 or 7, wherein the 5′ mutation is an exon 2 duplication.

9. The method of claim 8, wherein the administering is via a systemic route.

10. The method of claim 9, wherein the systemic route is by injection, infusion or implantation.

11. The method of any one of claims 5-10, wherein the PMO or PPMO is administered to the subject at a dose of about 1 to about 100 mg/kg.

12. The method of any one of claims 5-11, wherein the muscular dystrophy is Duchenne Muscular Dystrophy or Becker Muscular Dystrophy.

13. The method of any one of claims 5-12, wherein the level of functional dystrophin gene expression or protein expression in a cell of the subject is increased after administering the oligonucleotide, the antisense oligonucleotide construct, or the composition as compared to the level of functional dystrophin gene expression or protein expression before administering the oligonucleotide, the antisense oligonucleotide construct, or the composition.

14. The method of claim 13, wherein expression of functional dystrophin in the cell is detected by measuring the dystrophin protein level by Western blot, immunofluorescence, or immunohistochemistry in muscle biopsied before and after administering the oligonucleotide, the antisense oligonucleotide construct, or the composition.

15. The method of any one of claims 5-12, wherein the level of serum creatinine kinase is decreased after administering the oligonucleotide, the antisense oligonucleotide construct, or the composition as compared to the level of serum creatinine kinase before administering the oligonucleotide, the antisense oligonucleotide construct, or the composition.

16. The method of any one of claims 5-12 which results in improved muscle strength, improved muscle function, improved mobility, improved stamina, or a combination of two or more thereof in the subject.

17. The method of any one of claims 5-12, wherein muscular dystrophy progression in the subject is delayed or wherein muscle function in the subject is improved after administering the oligonucleotide, the antisense oligonucleotide construct, or the composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

18. The method of any one of claims 5-12 further comprising administering a second or combination therapy.

19. The method of claim 18 comprising administering a glucocorticoid.

20. Use of the
(a) the oligonucleotide of claim 1;
(b) the antisense oligonucleotide construct of any one of claims 2 and 3; or
(c) the composition of claim 4
for the preparation of a medicament for the treatment of a muscular dystrophy, or
for treating a muscular dystrophy in a subject in need thereof.

21. The use of claim 20, wherein treating is via a systemic route.

22. The use of claim 21, wherein the systemic route is by injection, infusion or implantation.

23. The use of any one of claims 20-22, wherein the PMO or PPMO is administered to the subject at a dose of about 1 to about 100 mg/kg.

24. The use of any one of claims 20-23, wherein the muscular dystrophy is Duchenne Muscular Dystrophy or Becker Muscular Dystrophy.

25. The use of any one of claims 20-24, wherein the level of functional dystrophin gene expression or protein expression in a cell of the subject is increased after use of the PMO or the PPMO, or the composition as compared to the level of functional dystrophin gene expression or protein expression before the use of the PMO or the PPMO, or the composition.

26. The use of claim 25, wherein expression of functional dystrophin in the cell is detected by measuring the dystrophin protein level by Western blot, immunofluorescence, or immunohistochemistry in muscle biopsied before and after administering the PMO or the PPMO, or the composition.

27. The use of any one of claims 20-24, wherein the level of serum creatinine kinase is decreased after administering the PMO or PPMO, or the composition as compared to the level of serum creatinine kinase before administering the PMO or PPMO, or the composition.

28. The use of any one of claims 20-24 which results in improved muscle strength, improved muscle function, improved mobility, improved stamina, or a combination of two or more thereof in the subject.

29. The use of any one of claims 20-24, wherein muscular dystrophy progression in the subject is delayed or wherein muscle function in the subject is improved after administering the PMO or PPMO, or the composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

30. The use of any one of claims 20-24 further comprising the use of a second or combination therapy.

31. The use of claim 30 comprising the use of a glucocorticoid.
